(19) United States
(12) Patent Application Publication
(10) Pub. No.: US 2025/0382673 A1
(43) Pub. Date: Dec. 18, 2025

(54) BIOMARKERS IN COLORECTAL CANCER

(71) Applicant: City of Hope, Duarte, CA (US)

(72) Inventors: Ajay Goel, Duarte, CA (US); Kota Nakamura, Duarte, CA (US)

(21) Appl. No.: 18/877,369

(22) PCT Filed: Jul. 3, 2023
(86) PCT No.: PCT/US2023/026836

§ 371 Date: Dec. 20, 2024

Related U.S. Application Data
(60) Provisional application No. 63/357,870, filed on Jul. 1, 2022.
(60) Provisional application No. 63/359,273, filed on Jul. 8, 2022.

(51) Int. Cl. C12Q 1/6886; C12Q 1/6809

(52) U.S. Cl. CPC C12Q 1/6886; C12Q 1/6809; C12Q 2600/106; C12Q 2600/158; C12Q 2600/178

(57) ABSTRACT

Provided herein, inter alia, are methods of treating colorectal cancer, diagnosing colorectal cancer, and monitoring colorectal cancer using biomarkers, such as miRNA, such as miR-513a-5p, miR-628-3p, miR-193a-5p, miR-210, miR-4304, miR-194-3p, miR-4453, or a combination of two or more thereof.

### CROSS REFERENCE TO RELATED APPLICATIONS

This application claims the benefit of priority to U.S. Application No. 63/359,273 filed Jul. 8, 2022, and U.S. Application No. 63/357,870 filed Jul. 1, 2022, the disclosures of which are incorporated by reference herein in their entirety.

### STATEMENT AS TO RIGHTS TO INVENTIONS MADE UNDER FEDERALLY SPONSORED RESEARCH AND DEVELOPMENT

This invention was made with government support under CA072851 and CA184792 awarded by the National Institutes of Health. The government has certain rights in the invention.

### BACKGROUND

Colorectal cancer (CRC) ranks third in cancer incidence and remains the third-leading cause of cancer-related deaths worldwide, in both men and women. In recent years, the incidence of sporadic CRC and associated mortality has declined worldwide. These improvements are likely due to the increased efforts in CRC screening that have allowed timely detection and removal of premalignant lesions and early-stage cancers, as well as improvements in treatment modalities. Unfortunately, data from epidemiological studies have revealed a significant trend for a rise in CRC incidence among individuals younger than 50 years of age who do not possess familial or hereditary disposition for this malignancy. This disease, referred to as the early-onset colorectal cancer (EOCRC), currently accounts for 10-15% of all new CRC diagnoses. At the current rate, it is estimated that the incidence for EOCRC is likely to double by the year 2030. While the reasons for this concerning trend in the younger population are poorly understood, there is a general consensus that patients with EOCRC are epidemiologically, biologically and pathologically distinct vis-à-vis those with late-onset colorectal cancer (LOCRC; patients ≥50 years old). Accordingly, for further characterization of EOCRC, these patients must be explored, assessed and clinically managed distinctly from those with LOCRC.

Notably, recent studies have suggested that patients with EOCRC have distinct clinical behaviors compared to those with LOCRC. EOCRC patients are more likely to exhibit an advanced stage tumor at initial presentation, distal tumor localization, signet ring histology, and a disease presentation with concurrent metastasis. This raises the logistical clinical concern that, since the tumors in EOCRC patients are often more aggressive than those with LOCRC, a delayed diagnosis could have a significant adverse impact and can lead to early death. Given the earlier age of onset and increased disease severity, these data highlight the need to develop screening approaches that can facilitate earlier detection and timely intervention for improving the overall survival in patients afflicted with EOCRC.

Recently, the guideline for initial CRC screening in the general population was lowered to 45 years by the American Cancer Society. However, this screening approach can benefit only a small fraction of younger individuals, as the overwhelming majority of these are likely not to have sporadic EOCRC, but are more likely to be those with a familial or hereditary form of CRC (e.g., Lynch syndrome). From a practical standpoint, lowering the age for CRC screening to capture patients with EOCRC using conventional colonoscopy has its challenges, as this procedure is invasive, has attendant risks, and is cost prohibitive for implementation in the average risk-population. In addition, currently available non-invasive tests including fecal and blood tests lack adequate diagnostic performance for the early detection of CRC, especially EOCRC, as these assays have yet to be explored or developed in this population. These limitations highlight the imperative need to develop robust, non-invasive biomarkers that can help overcome the challenges of the current generation of diagnostic assays, and facilitate the identification of patients with EOCRC. The present disclosure is directed to these important needs.

### BRIEF SUMMARY

Provided herein are methods of treating colorectal cancer in a patient in need thereof by: (a) administering to the patient an effective amount of an anti-cancer agent, (b) administering to the patient an effective amount of radiation therapy, (c) administering to the patient image-based screening, (d) surgically removing all or a portion of the colon of the patient, or (e) a combination of two or more thereof; wherein a biological sample obtained from the patient comprises an elevated expression level, relative to a control, of a RNA; wherein the RNA comprises miR-513a-5p, miR-628-3p, miR-193a-5p, miR-210, miR-4304, miR-194-3p, miR-4453, or a combination of two or more thereof.

Provided herein are methods of treating colorectal cancer in a patient in need thereof by: (i) detecting an elevated expression level, relative to a control, of RNA in a biological sample obtained from the patient; wherein the RNA comprises miR-513a-5p, miR-628-3p, miR-193a-5p, miR-210, miR-4304, miR-194-3p, miR-4453, or a combination of two or more thereof; and (ii) administering to the patient an effective amount of an anti-cancer agent, administering to the patient an effective amount of radiation therapy, administering to the patient image-based screening, surgically removing all or a portion of the colon of the patient, or a combination of two or more thereof.

Provided herein are methods of diagnosing a patient with colorectal cancer by: (i) detecting the expression level of RNA in a biological sample obtained from the patient; and (ii) diagnosing the patient as having colorectal cancer when the biological sample has an elevated expression level, relative to a control, of the RNA; wherein the RNA comprises miR-513a-5p, miR-628-3p, miR-193a-5p, miR-210, miR-4304, miR-194-3p, miR-4453, or a combination of two or more thereof.

Provided herein are methods of monitoring treatment in a patient having colorectal cancer or monitoring risk for developing colorectal cancer in a patient by: (i) detecting the expression level of RNA in a biological sample obtained from the patient at a first time point; (ii) detecting the expression level of the RNA in a biological sample obtained from the patient at a second time point, wherein the second time point is later than the first time point; and (iii) comparing the expression level of the RNA at the second time point to the expression level of the RNA at the first time point, thereby monitoring treatment or risk; wherein the RNA comprises miR-513a-5p, miR-628-3p, miR-193a-5p, miR-210, miR-4304, miR-194-3p, miR-4453, or a combination of two or more thereof.

Provided herein are methods of detecting RNA in a patient with colorectal cancer by detecting an elevated expression level, relative to a control, of RNA in a biological sample obtained from the patient; wherein the RNA comprises miR-513a-5p, miR-628-3p, miR-193a-5p, miR-210, miR-4304, miR-194-3p, miR-4453, or a combination of two or more thereof.

These and other embodiments of the disclosure are described herein.

### BRIEF DESCRIPTION OF THE DRAWINGS

FIGS. 1A-1C show differentially expressed miRNAs in patients with early stage EOCRC vs. normal mucosa specimens. FIG. 1A: The overall workflow for this study. FIG. 1B: A volcano plot illustrating miRNAs significantly upregulated in EOCRC (stage I-II) vs. normal mucosa specimens. FIG. 1C: Receiver operating characteristics (ROC) curve analysis with the selected 7 candidate miRNAs for discriminating stage I/II EOCRC tumors. * P<0.05

FIGS. 2A-2D show diagnostic performance of the miRNA panel in EOCRC vs. non-disease controls. FIG. 2A: The ROC curves analysis for a 4-miRNA panel in the training cohort. FIG. 2B: Risk score distribution plot in the training cohort. FIG. 2C: ROC curves analysis for a 4-miRNA panel in the validation cohort. FIG. 2D: Risk score distribution plot in the validation cohort.

FIGS. 3A-3C provide a diagnostic evaluation of the miRNA biomarker panel to identify different stages of EOCRC patients. FIG. 3A: ROC curve analysis to identify early stage (I and II) and late stage (III and IV) patients with EOCRC from non-disease controls in the validation cohort. FIG. 3B: Risk score analysis based on risk prediction formulae in early vs. late stage EOCRC patients and non-disease control in the validation cohort. FIG. 3C: Decision curve analysis to evaluate the performance of the miRNA panel.

FIGS. 4A-4E provide an evaluation of the miRNA panel in pre vs. post-operative blood specimens. FIGS. 4A-4D: Expression of miR-193a-5p, miR-210, miR-513a-5p, and miR-628-3p, respectively, in pre- and post-operative plasma specimens from EOCRC patients (where the y-axis is log-fold change). FIG. 4E: Assessment of risk probability based on risk prediction formula between pre- and postoperative EOCRC specimens.

FIG. 5 provides a ROC curve analysis to identify LOCRC patients from age-matched non-disease controls (≥50 years old).

FIG. 6 shows the miRNA-mRNA network analysis of miR-513a-5p, miR-628-3p, miR-193a-5p, and miR-210.

### DETAILED DESCRIPTION

Unless defined otherwise, technical and scientific terms used herein have the same meaning as commonly understood by a person of ordinary skill in the art. See, e.g., Singleton et al., Dictionary of Microbiology and Molecular Biology, 2nd ed., J. Wiley & Sons (New York, NY 1994); Sambrook et al., Molecular Cloning, A Laboratory Manual, Cold Springs Harbor Press (Cold Springs Harbor, NY 1989). Any methods, devices and materials similar or equivalent to those described herein can be used in the practice of this disclosure. The following definitions are provided to facilitate understanding of certain terms used frequently herein and are not meant to limit the scope of the present disclosure.

“Colorectal cancer” or “CRC” refers to a cancer that generally begins as growth (e.g., polyp) on the inner lining of the colon or rectum. Over time, the polyps can grow into the wall of the colon or rectum and into blood vessels or lymph nodes. The stage (extent of spread) of a colorectal cancer depends on how deeply it grows into the wall and if it has spread outside the colon or rectum. Colorectal cancer generally occurs when the patient is at least 50 years old, in which case it can also be referred to as late-onset colorectal cancer (LOCRC). The term “colorectal cancer” encompasses colon cancer and rectal cancer.

“Early-onset colorectal cancer” or “EOCRC” refers to colorectal cancer in a patient less than 50 years old. In embodiments, the patient is less than 50 years old and does not have a familial or hereditary disposition for colorectal cancer.

Methods for treating colorectal cancer, including early-onset colorectal cancer and late-onset colorectal cancer, include: (a) administering to the patient an effective amount of an anti-cancer agent, (b) administering to the patient an effective amount of radiation therapy, (c) administering to the patient image-based screening, (d) surgically removing all or a portion of the colon of the patient, or (e) a combination of two or more thereof. Surgery to remove all or portion of the colon of the patient are known in the art and include, for example, polypectomy, local excision via colonoscope, transanal excision (TAE), transanal endoscopic microsurgery (TEM), low anterior resection (LAR), proctectomy, abdominoperineal resection (APR), pelvic exenteration, and the like. The term “removing all or a portion of the colon” includes: (i) removing all or a portion of the colon, (ii) removing all or a portion of the rectum, and (iii) removing all or a portion of the rectum and all or a portion of the colon.

In “Stage 1” colorectal cancer, the cancer has grown through the muscularis mucosa into the submucosa (T1), and it may also have grown into the muscularis propria (T2), but it has not spread to nearby lymph nodes (N0) or to distant sites (M0).

“Stage 2” colorectal cancer is generally identified by one of the following: (i) the cancer has grown into the outermost layers of the colon or rectum but has not gone through them (T3); it has not reached nearby organs; and it has not spread to nearby lymph nodes (N0) or to distant sites (M0); (ii) the cancer has grown through the wall of the colon or rectum but has not grown into other nearby tissues or organs (T4a), and has not yet spread to nearby lymph nodes (N0) or to distant sites (M0); or (iii) the cancer has grown through the wall of the colon or rectum and is attached to or has grown into other nearby tissues or organs (T4b), but it has not yet spread to nearby lymph nodes (N0) or to distant sites (M0).

“Stage 3” colorectal cancer is generally identified by one of the following: (i) the cancer has grown through the mucosa into the submucosa (T1), and it may also have grown into the muscularis propria (T2); it has spread to 1 to 3 nearby lymph nodes (N1) or into areas of fat near the lymph nodes but not the nodes themselves (N1c); and it has not spread to distant sites (M0); (ii) the cancer has grown through the mucosa into the submucosa (T1); it has spread to 4 to 6 nearby lymph nodes (N2a); and it has not spread to distant sites (M0); (iii) the cancer has grown into the outermost layers of the colon or rectum (T3) or through the visceral peritoneum (T4a) but has not reached nearby organs; it has spread to 1 to 3 nearby lymph nodes (N1a or N1b) or into areas of fat near the lymph nodes but not the nodes themselves (N1c); and it has not spread to distant sites (M0); (iv) the cancer has grown into the muscularis propria (T2) or into the outermost layers of the colon or rectum (T3); it has spread to 4 to 6 nearby lymph nodes (N2a); and it has not spread to distant sites (M0); (v) the cancer has grown through the mucosa into the submucosa (T1), and it might also have grown into the muscularis propria (T2); it has spread to 7 or more nearby lymph nodes (N2b); and it has not spread to distant sites (M0); (vi) the cancer has grown through the wall of the colon or rectum (including the visceral peritoneum) but has not reached nearby organs (T4a); it has spread to 4 to 6 nearby lymph nodes (N2a); and it has not spread to distant sites (M0); (vi) the cancer has grown into the outermost layers of the colon or rectum (T3) or through the visceral peritoneum (T4a) but has not reached nearby organs; it has spread to 7 or more nearby lymph nodes (N2b); and it has not spread to distant sites (M0); or (viii) the cancer has grown through the wall of the colon or rectum and is attached to or has grown into other nearby tissues or organs (T4b); it has spread to at least one nearby lymph node or into areas of fat near the lymph nodes (N1 or N2); and it has not spread to distant sites (M0).

“Stage 4” colorectal cancer is generally identified by one of the following: (i) the cancer may or may not have grown through the wall of the colon or rectum (Any T); it might or might not have spread to nearby lymph nodes (Any N); it has spread to 1 distant organ (such as the liver or lung) or distant set of lymph nodes, but not to distant parts of the peritoneum (the lining of the abdominal cavity) (M1a); (ii) the cancer might or might not have grown through the wall of the colon or rectum (Any T); it might or might not have spread to nearby lymph nodes (Any N); it has spread to more than 1 distant organ (such as the liver or lung) or distant set of lymph nodes, but not to distant parts of the peritoneum (the lining of the abdominal cavity) (M1b); or (iii) the cancer might or might not have grown through the wall of the colon or rectum (Any T); it might or might not have spread to nearby lymph nodes (Any N); it has spread to distant parts of the peritoneum (the lining of the abdominal cavity), and may or may not have spread to distant organs or lymph nodes (M1c).

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). In embodiments, “nucleic acid” does not include nucleosides. The terms “polynucleotide,” “oligonucleotide,” “oligo” or the like refer, in the usual and customary sense, to a linear sequence of nucleotides. The term “nucleoside” refers, in the usual and customary sense, to a glycosylamine including a nucleobase and a five-carbon sugar (ribose or deoxyribose). Non limiting examples, of nucleosides include, cytidine, uridine, adenosine, guanosine, thymidine and inosine. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides, contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

A “microRNA,” “microRNA nucleic acid sequence,” “miR,” “miRNA” as used herein, refers to a nucleic acid that functions in RNA silencing and post-transcriptional regulation of gene expression. The term includes all forms of a miRNA, such as the pri-, pre-, and mature forms of the miRNA. In embodiments, microRNAs (miRNAs) are short (20-24 nt) non-coding RNAs that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce an approximately 70-nt stem-loop precursor miRNA (pre-miRNA), which is further cleaved by the cytoplasmic Dicer ribonuclease to generate the mature miRNA and antisense miRNA star (miRNA*) products. The mature miRNA is incorporated into a RNA-induced silencing complex (RISC), which recognizes target mRNAs through imperfect base pairing with the miRNA and most commonly results in translational inhibition or destabilization of the target mRNA.

Nucleic acids can include nonspecific sequences. As used herein, the term “nonspecific sequence” refers to a nucleic acid sequence that contains a series of residues that are not designed to be complementary to or are only partially complementary to any other nucleic acid sequence. By way of example, a nonspecific nucleic acid sequence is a sequence of nucleic acid residues that does not function as an inhibitory nucleic acid when contacted with a cell or organism.

The term “complement,” as used herein, refers to a nucleotide (e.g., RNA or DNA) or a sequence of nucleotides capable of base pairing with a complementary nucleotide or sequence of nucleotides. As described herein and commonly known in the art the complementary (matching) nucleotide of adenosine is thymidine and the complementary (matching) nucleotide of guanosine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and a non-coding sequences, wherein the non-coding sequence contains complementary nucleotides to the coding sequence and thus forms the complement of the coding sequence.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding RNA that is present within the cell or the amount of protein encoded by that DNA produced by the cell. The level of expression of non-coding nucleic acid molecules (e.g., miRNA, mRNA) may be detected by standard PCR or Northern blot methods well known in the art.

The terms “expression level,” “amount,” or “level” of a biomarker is a detectable level in a biological sample. “Expression” generally refers to the process by which information (e.g., gene-encoded and/or epigenetic) is converted into the structures present and operating in the cell. Therefore, “expression” may refer to transcription into a polynucleotide, translation into a polypeptide, or even polynucleotide and/or polypeptide modifications (e.g., posttranslational modification of a polypeptide). Fragments of the transcribed polynucleotide, the translated polypeptide, or polynucleotide and/or polypeptide modifications (e.g., post-translational modification of a polypeptide) shall also be regarded as expressed whether they originate from a transcript generated by alternative splicing or a degraded transcript, or from a post-translational processing of the polypeptide, e.g., by proteolysis. “Expressed genes” include those that are transcribed into a polynucleotide as mRNA and then translated into a polypeptide, and also those that are transcribed into RNA but not translated into a polypeptide (for example, miRNA, transfer RNA, ribosomal RNA, lncRNA). Expression levels can be measured by methods known to one skilled in the art and also disclosed herein. The expression level or amount of a biomarker (e.g., RNA, miRNA) can be used to diagnose and/or treat a subject with colorectal cancer.

The terms an “elevated expression level” or “elevated level” of gene expression is an expression level of the gene that is higher than the expression level of the gene in a control. The control may be any suitable control, as described herein. In embodiments, an “elevated expression level” of the biomarker gene compared to the control (when the expression level of the biomarker is greater than the corresponding control) is, for example, an increase in the expression level of about 10%, 20%, 30%, 40%, 50%, 60%, 70%, 80%, 90%, 95%, 96%, 97%, 98% or 99% or greater relative to the control. In embodiments, an “elevated expression level” of the biomarker gene is an amount that is statistically significantly greater than the expression level of the control.

An expression level of the gene that is “about the same as” the expression level of the gene in a control or in a comparison to a previous gene expression level (e.g., in the case of monitoring a gene expression level at different time points). The control may be any suitable control, as described herein. In embodiments, “about the same as” is +/−25% of the expression level of the biomarker gene in a control or at a previous time point. In embodiments, “about the same as” is +/−20% of the expression level of the biomarker gene in a control or at a previous time point. In embodiments, “about the same as” is +/−15% of the expression level of the biomarker gene in a control or at a previous time point. In embodiments, “about the same as” is +/−10% of the expression level of the biomarker gene in a control or at a previous time point. In embodiments, “about the same as” is +/−5% of the expression level of the biomarker gene in a control or at a previous time point. In embodiments, an expression level of the biomarker gene that is “about the same as” a control or a previous time point is an amount that is not statistically significantly different than the expression level of the control or the previous time point.

The terms “biomarker gene” and “biomarker” are used interchangeably and in accordance with their plain and ordinary meaning. In embodiments, a biomarker is a gene or a set of genes (i.e., a biomarker gene). Biomarkers include, but are not limited to, polynucleotides (e.g., DNA, and/or RNA), polynucleotide copy number alterations (e.g., DNA copy numbers), polypeptides, or polypeptide and polynucleotide modifications (e.g., posttranslational modifications). In embodiments, a biomarker refers to RNA (e.g., miRNA), the expression level of which is associated with colorectal cancer. In embodiments, a biomarker refers to miRNA, the expression level of which is associated with colorectal cancer.

Biomarker levels may be detected at either the protein or gene expression level. Proteins expressed by biomarkers can be quantified by immunohistochemistry (IHC) or flow cytometry with an antibody that detects the proteins. Biomarker expression can be quantified by multiple platforms such as real-time polymerase chain reaction (rtPCR), NanoString, RNAseq, or in situ hybridization. There is a range of biomarker expression across as measured by NanoString. In embodiments, quantitative rtPCR, NanoString, RNAseq, and in situ hybridization are platforms to quantitate biomarker gene expression. For NanoString, RNA is extracted from a biological sample and a known quantity of RNA is placed on the NanoString machine for gene expression detection using gene specific probes. The number of counts of biomarkers within a sample is determined and normalized to a set of housekeeping genes. To determine a threshold for increased or decreased biomarker levels, one skilled in the art could assess biomarker levels in a control group of samples and select the 10th, 20th, 25th, 30th, 40th, 50th, 60th, 70th, 75th, 80th or 90th percentile of biomarker gene expression. In embodiments, the increased or decreased expression of biomarkers may be determined by calculating the H-score for the expression of the biomarkers. Thus, the increased or decreased expression of biomarkers may have an H-score. As used herein, an “H-score” or “Histoscore” is a numerical value determined by a semi-quantitative method commonly known for immunohistochemically evaluating protein expression in tumor samples. The H-score may be calculated using the following formula: [1×(% cells 1+)+2×(% cells 2+)+3×(% cells 3+)]. According to this formula, the H-score is calculated by determining the percentage of cells having a given staining intensity level (i.e., level 1+, 2+, or 3+ from lowest to highest intensity level), weighting the percentage of cells having the given intensity level by multiplying the cell percentage by a factor (e.g., 1, 2, or 3) that gives more relative weight to cells with higher-intensity membrane staining, and summing the results to obtain a H-score. Commonly H-scores range from 0 to 300. Further description on the determination of H-scores in tumor cells can be found in Hirsch et al, J Clin Oncol 21:3798-3807, 2003 and John et al, Oncogene 28: S14-S23, 2009. IHC or other methods known in the art may be used for detecting biomarker expression.

“Control” is used in accordance with its plain ordinary meaning and refers to an assay, comparison, or experiment in which the subjects or reagents of the experiment are treated as in a parallel experiment except for omission of a procedure, reagent, or variable of the experiment. In embodiments, the control is used as a standard of comparison in evaluating experimental effects. In embodiments, a control is the measurement of the activity or level of RNA. In embodiments, a control is a healthy patient or a healthy population of patients. In embodiments, a control is an average value from a population of similar patients, e.g., healthy patients with a similar medical background, age, weight, etc. In embodiments, the control is a healthy patient or a population of healthy patients. In embodiments, a healthy patient can be referred to as a non-diseased patient or non-diseased control. In embodiments, the control is a population of non-diseased patients. In embodiments, a non-diseased patient is a patient that does not have cancer. In embodiments, a non-diseased patient is a patient that does not have colorectal cancer. In embodiments, the control is a patient that does not have cancer or a population of patients that do not have cancer. In embodiments, the control is a patient that does not have colorectal cancer or a population of patients that do not have colorectal cancer. In embodiments, the control is a patient that does not have colorectal or a population of patients that do not have colorectal. In embodiments, the control is an average value from population of healthy patients. A control can also be obtained from the same patient, e.g., from an earlier-obtained sample, prior to disease, or prior to treatment. One of skill will recognize that controls can be designed for assessment of any number of parameters. In embodiments, a control is a negative control. In embodiments, such as some embodiments relating to detecting the level of expression of a gene/protein or a subset of genes/proteins, a control comprises the average amount of expression (e.g., protein or mRNA) in a population of subjects (e.g., with cancer) or in a healthy or general population. In embodiments, the control comprises an average amount (e.g. amount of expression) in a population in which the number of subjects (n) is 5 or more, 20 or more, 50 or more, 100 or more, 1,000 or more, and the like. In embodiments, the control is a standard control. In embodiments, a standard control is a level of expression of the biomarker (e.g., RNA, miRNA) that has been correlated with the diagnosis of colorectal cancer in a subject. In embodiments, a standard control is a level of expression of the biomarker (e.g., RNA, miRNA) that has been correlated with a healthy subject (i.e., a subject that does not have colorectal cancer). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “healthy patient” refers to a non-diseased patient. In embodiments, a healthy patient is a patient that does not have cancer. In embodiments, a healthy patient is a patient that does not have colorectal cancer (e.g., EOCRC or LOCRC). In embodiments, a healthy patient is a patient that does not have early-onset colorectal cancer In embodiments, a healthy patient is a patient that does not have late-onset colorectal cancer

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid including two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein including two or more subsequences that are not found in the same relationship to each other in nature (e.g., a fusion protein).

The phrase “specifically (or selectively) binds” to an antibody or “specifically (or selectively) immunoreactive with,” when referring to a protein or peptide refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

The terms “isolate” or “isolated”, when applied to a nucleic acid, virus, or protein, denotes that the nucleic acid, virus, or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. An RNA that is the predominant species present in a preparation is substantially purified.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (e.g., www.ncbi.nlm.nih.gov/BLAST/or the like). Such sequences are then the to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

As used herein, the term “about” means a range of values including the specified value, which a person of ordinary skill in the art would consider reasonably similar to the specified value. In embodiments, “about” means within a standard deviation using measurements generally acceptable in the art. In embodiments, “about” means a range extending to +/−10% of the specified value. In embodiments, “about” includes the specified value.

The singular terms “a,” “an,” and “the” include the plural reference unless the context clearly indicates otherwise.

A “therapeutic agent” or “anticancer agent” as used herein refer to an agent (e.g., compound, pharmaceutical composition) that when administered to a subject will have the intended therapeutic effect, e.g., treatment or amelioration of colorectal cancer, or their symptoms including any objective or subjective parameter of treatment such as abatement; remission; diminishing of symptoms or making the cancer more tolerable to the patient; slowing in the rate of degeneration or decline; making the final point of degeneration less debilitating; or improving a patient's physical or mental well-being.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. In embodiments, a biological sample is tissue. In embodiments, a biological sample is blood. In embodiments, a biological sample is a serum sample (e.g., the fluid and solute component of blood without the clotting factors). In embodiments, a biological sample is a plasma sample (e.g, the liquid portion of blood).

“Liquid biological sample” refers to liquid materials obtained or derived from a subject or patient. Liquid biological samples include bodily fluids such as blood and blood fractions or products (e.g., serum, plasma, platelets, red blood cells, and the like), sputum, urine, synovial fluid, and the like. In embodiments, a liquid biological sample is a blood sample.

The term “diagnosis” is used in accordance with its plain and ordinary meaning and refers to an identification or likelihood of the presence of a disease (e.g., colorectal cancer) or outcome in a subject.

“Image-based screening” refers to methods using imaging technology to detect a cancer or tumor in a patient. Exemplary types of image-based screening include x-rays, computed tomography (CT), magnetic resonance imaging (MRI), positron emission tomography (PET), and ultrasound. In embodiments, the image-based screening is CT, MRI, or ultrasound. In embodiments, the ultrasound is endoscopic ultrasonography (EUS). In embodiments, the image-based screening is CT, MRI, or EUS. In embodiments, the image-based screening is MRI or EUS. In embodiments, the image-based screening is CT. In embodiments, the image-based screening is MRI. In embodiments, the image-based screening is EUS.

The terms “treating” or “treatment” are used in accordance with their plain and ordinary meaning and broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, delay or slowing of disease progression, amelioration or palliation of the disease state, and remission, whether partial or total and whether detectable or undetectable. Treatment may inhibit the disease's spread; relieve the disease's symptoms, fully or partially remove the disease's underlying cause, shorten a disease's duration, or do a combination of these things. Treatment methods include administering to a subject a therapeutically effective amount of an active agent. The term “treating” does not including preventing.

The term “preventing” or “prevent” is used in accordance with its plain and ordinary meaning and refers to a decrease in the occurrence of disease symptoms in a patient or to keep a disease from occurring. The prevention may be complete (no detectable symptoms) or partial, such that fewer symptoms are observed than would likely occur absent treatment.

An “effective amount” is an amount sufficient to accomplish a stated purpose (e.g. achieve the effect for which it is administered, treat a disease). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). The exact amounts will depend on the purpose of the treatment, and will be ascertainable by one skilled in the art using known techniques. In embodiments, “therapeutically effective amount” refers to the amount of the therapeutic agent sufficient to treat or ameliorate colorectal cancer, as described above. For any therapeutic agent described herein, the therapeutically effective amount can be initially determined from cell culture assays. Target concentrations will be those concentrations of active compound(s) that are capable of achieving the methods described herein, as measured using the methods described herein or known in the art. As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan. Dosages may be varied depending upon the requirements of the patient and the therapeutic agent being employed. The dose administered to a patient should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state. A “therapeutically effective amount” can also be found on the label or Prescribing Information for commercially available therapeutic agents.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

The terms “patient” or “subject” are used in accordance with its plain and ordinary meaning and refer to a living organism suffering from or prone to a disease that can be treated by administration of a pharmaceutical composition, such as anti-cancer agents and chemotherapeutic agents. Non-limiting examples include humans, other mammals, bovines, rats, mice, dogs, cats, monkeys, and other non-mammalian animals. In embodiments, a patient is human patient. In embodiments, the human patient is less than 50 years old. In embodiments, the human patient is less than 45 years old. In embodiments, the human patient is less than 40 years old. In embodiments, the human patient has Lynch syndrome (hereditary nonpolyposis colorectal cancer).

The terms “metastasis,” “metastatic,” and “metastatic cancer” can be used interchangeably and refer to the spread of a proliferative disease or disorder, e.g., cancer, from one organ or another non-adjacent organ or body part. Cancer occurs at an originating site, e.g., pancreas, which site is referred to as a primary tumor, e.g., primary colorectal cancer. Some cancer cells in the primary tumor or originating site acquire the ability to penetrate and infiltrate surrounding normal tissue in the local area and/or the ability to penetrate the walls of the lymphatic system or vascular system circulating through the system to other sites and tissues in the body. A second clinically detectable tumor formed from cancer cells of a primary tumor is referred to as a metastatic or secondary tumor. When cancer cells metastasize, the metastatic tumor and its cells are presumed to be similar to those of the original tumor. Thus, if colorectal cancer metastasizes to the lymph nodes, the secondary tumor at the site of the lymph nodes consist of colorectal cancer cells and not abnormal lymph node cells. The secondary tumor in the lymph nodes is referred to as lymph node metastasis. Thus, the phrase metastatic cancer refers to a disease in which a subject has or had a primary tumor and has one or more secondary tumors. The phrases non-metastatic cancer or subjects with cancer that is not metastatic refers to diseases in which subjects have a primary tumor but not one or more secondary tumors.

### Methods of Treatment

Provided herein are methods of treating colorectal cancer in a patient in need thereof. In embodiments, the methods of treating colorectal cancer in a patient in need thereof comprise administering to the patient an effective amount of an anti-cancer agent, administering to the patient an effective amount of radiation therapy, administering to the patient image-based screening, surgically removing all or a portion of the colon of the patient, or a combination of two or more thereof; wherein a biological sample obtained from the patient comprises an elevated expression level, relative to a control, of a RNA; wherein the RNA comprises miR-513a-5p, miR-628-3p, miR-193a-5p, miR-210, miR-4304, miR-194-3p, miR-4453, or a combination of two or more thereof. In embodiments, the methods of treating colorectal cancer in a patient in need thereof comprise administering to the patient an effective amount of an anti-cancer agent, administering to the patient an effective amount of radiation therapy, administering to the patient image-based screening, surgically removing all or a portion of the colon of the patient, or a combination of two or more thereof; wherein a biological sample obtained from the patient comprises an elevated expression level, relative to a control, of a RNA; wherein the RNA comprises miR-513a-5p, miR-628-3p, miR-193a-5p, miR-210, or a combination of two or more thereof. In embodiments, the methods of treating colorectal cancer in a patient in need thereof comprise: (i) detecting an elevated expression level, relative to a control, of RNA in a biological sample obtained from the patient; wherein the RNA comprises miR-513a-5p, miR-628-3p, miR-193a-5p, miR-210, miR-4304, miR-194-3p, miR-4453, or a combination of two or more thereof; and (ii) administering to the patient an effective amount of an anti-cancer agent, administering to the patient an effective amount of radiation therapy, administering to the patient image-based screening, surgically removing all or a portion of the colon of the patient, or a combination of two or more thereof. In embodiments, the methods of treating colorectal cancer in a patient in need thereof comprise: (i) detecting an elevated expression level, relative to a control, of RNA in a biological sample obtained from the patient; wherein the RNA comprises miR-513a-5p, miR-628-3p, miR-193a-5p, miR-210, or a combination of two or more thereof; and (ii) administering to the patient an effective amount of an anti-cancer agent, administering to the patient an effective amount of radiation therapy, administering to the patient image-based screening, surgically removing all or a portion of the colon of the patient, or a combination of two or more thereof. In embodiments, the methods comprise surgically removing all or a portion of the colon of the patient. In embodiments, the methods comprise administering to the patient an effective amount of an anti-cancer agent and surgically removing all or a portion of the colon of the patient. In embodiments, the methods comprise administering to the patient an effective amount of an anti-cancer agent. In embodiments, the methods comprise administering to the patient an effective amount of an anti-cancer agent, administering to the patient an effective amount of radiation therapy, and surgically removing all or a portion of the colon of the patient.

Provided herein are methods of diagnosing a patient with colorectal cancer. In embodiments, the methods of diagnosing a patient with colorectal cancer comprise: (i) detecting the expression level of RNA in a biological sample obtained from the patient; and (ii) diagnosing the patient as having colorectal cancer when the biological sample has an elevated expression level, relative to a control, of the RNA; wherein the RNA comprises miR-513a-5p, miR-628-3p, miR-193a-5p, miR-210, miR-4304, miR-194-3p, miR-4453, or a combination of two or more thereof. In embodiments, the methods of diagnosing a patient with colorectal cancer comprises: (i) detecting the expression level of RNA in a biological sample obtained from the patient; and (ii) diagnosing the patient as having colorectal cancer when the biological sample has an elevated expression level, relative to a control, of the RNA; wherein the RNA comprises miR-513a-5p, miR-628-3p, miR-193a-5p, miR-210, or a combination of two or more thereof. In embodiments, the methods further comprise administering to the patient an effective amount of an anti-cancer agent, administering to the patient an effective amount of radiation therapy, administering to the patient image-based screening, surgically removing all or a portion of the colon of the patient, or a combination of two or more thereof. In embodiments, the methods further comprise surgically removing all or a portion of the colon of the patient. In embodiments, the methods further comprise administering to the patient an effective amount of an anti-cancer agent and surgically removing all or a portion of the colon of the patient. In embodiments, the methods further comprise administering to the patient an effective amount of an anti-cancer agent. In embodiments, the methods further comprise administering to the patient an effective amount of an anti-cancer agent, administering to the patient an effective amount of radiation therapy, and surgically removing all or a portion of the colon of the patient.

Provided herein are methods of monitoring treatment in a patient having colorectal cancer. In embodiments, the methods of monitoring treatment in a patient having colorectal cancer comprise: (i) detecting the expression level of RNA in a biological sample obtained from the patient at a first time point; (ii) detecting the expression level of the RNA in a biological sample obtained from the patient at a second time point, wherein the second time point is later than the first time point; and (iii) comparing the expression level of the RNA at the second time point to the expression level of the RNA at the first time point, thereby monitoring treatment; wherein the RNA comprises miR-513a-5p, miR-628-3p, miR-193a-5p, miR-210, miR-4304, miR-194-3p, miR-4453, or a combination of two or more thereof. In embodiments, the methods of monitoring treatment in a patient having colorectal cancer comprise: (i) detecting the expression level of RNA in a biological sample obtained from the patient at a first time point; (ii) detecting the expression level of the RNA in a biological sample obtained from the patient at a second time point, wherein the second time point is later than the first time point; and (iii) comparing the expression level of the RNA at the second time point to the expression level of the RNA at the first time point, thereby monitoring treatment; wherein the RNA comprises miR-513a-5p, miR-628-3p, miR-193a-5p, miR-210, or a combination of two or more thereof. When the expression level of the RNA at the second time point is lower than the expression level of the RNA at the first time, the treatment is successful or providing positive results. If the treatment is successful or providing positive results, the physician can make a determination of whether to discontinue, modify, or continue with the existing treatment. In embodiments, the patient has a good prognosis when the expression level of the RNA at the second time point is lower than the expression level of the RNA at the first time. When the expression level of the RNA at the second time point is about the same as or greater than the expression level of the RNA at the first time, the treatment is unsuccessful. If the treatment is unsuccessful, the physician can make a determination of whether to modify or continue with the existing treatment. In embodiments, the patient has a poor prognosis when the expression level of the RNA at the second time point is about the same as or greater than the expression level of the RNA at the first time. In embodiments, the methods further comprise administering to the patient an effective amount of an anti-cancer agent, administering to the patient an effective amount of radiation therapy, administering to the patient image-based screening, surgically removing all or a portion of the colon of the patient, or a combination of two or more thereof after step (i) and before step (ii). In embodiments, the methods further comprise surgically removing all or a portion of the colon of the patient after step (i) and before step (ii). In embodiments, the methods further comprise administering to the patient an effective amount of an anti-cancer agent after step (i) and before step (ii). In embodiments, the methods further comprise surgically removing all or a portion of the colon of the patient and administering to the patient an effective amount of an anti-cancer agent after step (i) and before step (ii). In embodiments, step (i) occurs in a treatment naïve patient. In embodiments, step (i) occurs after a patient has been administered an effective amount of an anti-cancer agent, an effective amount of radiation therapy, image-based screening, surgical removal of all or a portion of the patient's colon, or a combination of two or more thereof. In embodiments, step (i) occurs after surgical removal of all or a portion of the patient's colon. In embodiments, step (i) occurs after a patient has been administered an effective amount of an anti-cancer agent and after surgical removal of all or a portion of the patient's colon. In embodiments, step (i) occurs after a patient has been administered an effective amount of an anti-cancer agent. In embodiments, step (i) occurs after a patient has been administered an effective amount of an anti-cancer agent, an effective amount of radiation therapy, and surgical removal of all or a portion of the patient's colon. In embodiments, the methods further comprise administering to the patient an effective amount of an anti-cancer agent, administering to the patient an effective amount of radiation therapy, administering to the patient image-based screening, surgically removing all or a portion of the colon of the patient, or a combination of two or more thereof. In embodiments, the methods further comprise surgically removing all or a portion of the colon of the patient. In embodiments, the methods further comprise administering to the patient an effective amount of an anti-cancer agent and surgically removing all or a portion of the colon of the patient. In embodiments, the methods further comprise administering to the patient an effective amount of an anti-cancer agent. In embodiments, the methods further comprise administering to the patient an effective amount of an anti-cancer agent, administering to the patient an effective amount of radiation therapy, and surgically removing all or a portion of the colon of the patient.

Provided herein are methods of monitoring risk for developing colorectal cancer in a patient. In embodiments, the methods of monitoring risk for developing colorectal cancer in a patient comprise: (i) detecting the expression level of RNA in a biological sample obtained from the patient at a first time point; (ii) detecting the expression level of the RNA in a biological sample obtained from the patient at a second time point, wherein the second time point is later than the first time point; and (iii) comparing the expression level of the RNA at the second time point to the expression level of the RNA at the first time point, thereby monitoring risk; wherein the RNA comprises miR-513a-5p, miR-628-3p, miR-193a-5p, miR-210, miR-4304, miR-194-3p, miR-4453, or a combination of two or more thereof. In embodiments, the methods of monitoring risk for developing colorectal cancer in a patient comprise: (i) detecting the expression level of RNA in a biological sample obtained from the patient at a first time point; (ii) detecting the expression level of the RNA in a biological sample obtained from the patient at a second time point, wherein the second time point is later than the first time point; and (iii) comparing the expression level of the RNA at the second time point to the expression level of the RNA at the first time point, thereby monitoring risk; wherein the RNA comprises miR-513a-5p, miR-628-3p, miR-193a-5p, miR-210, or a combination of two or more thereof. When the expression level of the RNA at the second time point is greater than the expression level of the RNA at the first time, the patient is at risk for developing colorectal cancer or the patient may have developed colorectal cancer. When the expression level of the RNA at the second time point is about the same as or less than the expression level of the RNA at the first time, the patient is not at risk for developing colorectal cancer or has not developed colorectal cancer. In embodiments, the methods further comprise administering to the patient image-based screening after step (i) and before step (ii).

Provided herein are methods of detecting RNA in a patient having colorectal cancer. In embodiments, the methods of detecting RNA in a patient having colorectal cancer comprise detecting an elevated expression level, relative to a control, of RNA in a biological sample obtained from the patient; wherein the RNA comprises miR-513a-5p, miR-628-3p, miR-193a-5p, miR-210, miR-4304, miR-194-3p, miR-4453, or a combination of two or more thereof. In embodiments, the methods of detecting RNA in a patient having colorectal cancer comprising detecting an elevated expression level, relative to a control, of RNA in a biological sample obtained from the patient; wherein the RNA comprises miR-513a-5p, miR-628-3p, miR-193a-5p, miR-210, or a combination of two or more thereof. In embodiments, the methods further comprise administering to the patient an effective amount of an anti-cancer agent, administering to the patient an effective amount of radiation therapy, administering to the patient image-based screening, surgically removing all or a portion of the colon of the patient, or a combination of two or more thereof. In embodiments, the methods further comprise surgically removing all or a portion of the colon of the patient. In embodiments, the methods further comprise administering to the patient an effective amount of an anti-cancer agent and surgically removing all or a portion of the colon of the patient. In embodiments, the methods further comprise administering to the patient an effective amount of an anti-cancer agent. In embodiments, the methods further comprise administering to the patient an effective amount of an anti-cancer agent, administering to the patient an effective amount of radiation therapy, and surgically removing all or a portion of the colon of the patient.

In embodiments of the methods described herein, the RNA comprises miR-513a-5p, miR-628-3p, miR-193a-5p, miR-210, miR-4304, miR-194-3p, miR-4453, or a combination of two or more thereof. In embodiments, the RNA comprises one RNA selected from the group consisting of miR-513a-5p, miR-628-3p, miR-193a-5p, miR-210, miR-4304, miR-194-3p, and miR-4453. In embodiments, the RNA comprises at least one RNA selected from the group consisting of miR-513a-5p, miR-628-3p, miR-193a-5p, miR-210, miR-4304, miR-194-3p, and miR-4453. In embodiments, the RNA comprises two RNA selected from the group consisting of miR-513a-5p, miR-628-3p, miR-193a-5p, miR-210, miR-4304, miR-194-3p, and miR-4453. In embodiments, the RNA comprises at least two RNA selected from the group consisting of miR-513a-5p, miR-628-3p, miR-193a-5p, miR-210, miR-4304, miR-194-3p, and miR-4453. In embodiments, the RNA comprises three RNA selected from the group consisting of miR-513a-5p, miR-628-3p, miR-193a-5p, miR-210, miR-4304, miR-194-3p, and miR-4453. In embodiments, the RNA comprises at least three RNA selected from the group consisting of miR-513a-5p, miR-628-3p, miR-193a-5p, miR-210, miR-4304, miR-194-3p, and miR-4453. In embodiments, the RNA comprises four RNA selected from the group consisting of miR-513a-5p, miR-628-3p, miR-193a-5p, miR-210, miR-4304, miR-194-3p, and miR-4453. In embodiments, the RNA comprises at least four RNA selected from the group consisting of miR-513a-5p, miR-628-3p, miR-193a-5p, miR-210, miR-4304, miR-194-3p, and miR-4453. In embodiments, the RNA comprises five RNA selected from the group consisting of miR-513a-5p, miR-628-3p, miR-193a-5p, miR-210, miR-4304, miR-194-3p, and miR-4453. In embodiments, the RNA comprises at least five RNA selected from the group consisting of miR-513a-5p, miR-628-3p, miR-193a-5p, miR-210, miR-4304, miR-194-3p, and miR-4453. In embodiments, the RNA comprises six RNA selected from the group consisting of miR-513a-5p, miR-628-3p, miR-193a-5p, miR-210, miR-4304, miR-194-3p, and miR-4453. In embodiments, the RNA comprises at least six RNA selected from the group consisting of miR-513a-5p, miR-628-3p, miR-193a-5p, miR-210, miR-4304, miR-194-3p, and miR-4453. In embodiments, the RNA comprises miR-513a-5p, miR-628-3p, miR-193a-5p, miR-210, miR-4304, miR-194-3p, and miR-4453. In embodiments, the RNA consists of miR-513a-5p, miR-628-3p, miR-193a-5p, miR-210, miR-4304, miR-194-3p, and miR-4453. With reference to this embodiment, “consists of” means that only the 7 named RNA (i.e., no other RNA) are used in the methods described herein.

In embodiments of the methods described herein, the RNA comprises miR-513a-5p, miR-628-3p, miR-193a-5p, miR-210, miR-4304, miR-194-3p, miR-4453, or a combination of two or more thereof. In embodiments, the RNA comprises miR-4304, miR-194-3p, miR-4453, or a combination of two or more thereof. In embodiments, the RNA comprises miR-4304, miR-194-3p, and miR-4453. In embodiments, the RNA comprises miR-4304. In embodiments, the RNA comprises miR-194-3p. In embodiments, the RNA comprises miR-4453. In embodiments, the RNA comprises miR-4304 and miR-194-3p. In embodiments, the RNA comprises miR-4304 and miR-4453. In embodiments, the RNA comprises miR-194-3p and miR-4453.

In embodiments of the methods described herein, the RNA comprises miR-513a-5p, miR-628-3p, miR-193a-5p, miR-210, or a combination of two or more thereof. In embodiments, the RNA comprises at least one RNA selected from the group consisting of miR-513a-5p, miR-628-3p, miR-193a-5p, and miR-210. In embodiments, the RNA comprises one RNA selected from the group consisting of miR-513a-5p, miR-628-3p, miR-193a-5p, and miR-210. In embodiments, the RNA comprises miR-513a-5p. In embodiments, the RNA comprises miR-628-3p. In embodiments, the RNA comprises miR-193a-5p. In embodiments, the RNA comprises miR-210. In embodiments, the RNA comprises at least two RNA selected from the group consisting of miR-513a-5p, miR-628-3p, miR-193a-5p, and miR-210. In embodiments, the RNA comprises two RNA selected from the group consisting of miR-513a-5p, miR-628-3p, miR-193a-5p, and miR-210. In embodiments, the RNA comprises miR-513a-5p and miR-628-3p. In embodiments, the RNA comprises miR-513a-5p and miR-193a-5p. In embodiments, the RNA comprises miR-513a-5p and miR-210. In embodiments, the RNA comprises miR-628-3p and miR-193a-5p. In embodiments, the RNA comprises miR-628-3p and miR-210. In embodiments, the RNA comprises miR-193a-5p and miR-210. In embodiments, the RNA comprises at least three RNA selected from the group consisting of miR-513a-5p, miR-628-3p, miR-193a-5p, and miR-210. In embodiments, the RNA comprises three RNA selected from the group consisting of miR-513a-5p, miR-628-3p, miR-193a-5p, and miR-210. In embodiments, the RNA comprises miR-513a-5p, miR-628-3p, and miR-193a-5p. In embodiments, the RNA comprises miR-513a-5p, miR-628-3p, and miR-210. In embodiments, the RNA comprises miR-513a-5p, miR-193a-5p, and miR-210. In embodiments, the RNA comprises miR-628-3p, miR-193a-5p, and miR-210. In embodiments, the RNA comprises miR-513a-5p, miR-628-3p, miR-193a-5p, and miR-210. In embodiments, the RNA consists of miR-513a-5p, miR-628-3p, miR-193a-5p, and miR-210. With reference to this embodiment, “consists of” means that only the 4 named RNA (i.e., no other RNA) are used in the methods described herein.

In embodiments of the methods described herein, the biological sample is any biological sample. In embodiments, the biological sample is a blood sample or a tissue sample. In embodiments, the biological sample is a tissue sample. In embodiments, the tissue sample is a tumor tissue sample. In embodiments, the biological sample is a stool sample. In embodiments, the biological sample is a liquid biological sample. In embodiments, the biological sample is a blood sample. In embodiments, the blood sample is a serum sample or a plasma sample. In embodiments, the biological sample is a serum sample. In embodiments, the biological sample is a plasma sample. In embodiments when the RNA comprises miR-513a-5p, miR-628-3p, miR-193a-5p, miR-210, miR-4304, miR-194-3p, miR-4453 or a combination of two or more thereof, the biological sample is a liquid biological sample. In embodiments when the RNA comprises miR-513a-5p, miR-628-3p, miR-193a-5p, miR-210, miR-4304, miR-194-3p, miR-4453 or a combination of two or more thereof, the biological sample is a blood sample. In embodiments when the RNA comprises miR-513a-5p, miR-628-3p, miR-193a-5p, miR-210, miR-4304, miR-194-3p, miR-4453 or a combination of two or more thereof, the biological sample is a tissue sample. In embodiments when the RNA comprises miR-513a-5p, miR-628-3p, miR-193a-5p, miR-210, miR-4304, miR-194-3p, miR-4453 or a combination of two or more thereof, the biological sample is a tumor tissue sample. In embodiments when the RNA comprises miR-513a-5p, miR-628-3p, miR-193a-5p, miR-210, or a combination of two or more thereof, the biological sample is a liquid biological sample. In embodiments when the RNA comprises miR-513a-5p, miR-628-3p, miR-193a-5p, miR-210, or a combination of two or more thereof, the biological sample is a blood sample. In embodiments when the RNA comprises miR-513a-5p, miR-628-3p, miR-193a-5p, miR-210, or a combination of two or more thereof, the biological sample is a serum sample. In embodiments when the RNA comprises miR-513a-5p, miR-628-3p, miR-193a-5p, miR-210, or a combination of two or more thereof, the biological sample is a plasma sample.

In embodiments of the methods described herein, the colorectal cancer is early-onset colorectal cancer or late-onset colorectal cancer. In embodiments, the colorectal cancer is early-onset colorectal cancer. In embodiments, the colorectal cancer is late-onset colorectal cancer. In embodiments, the colorectal cancer is Stage I or Stage II. In embodiments, the colorectal cancer is Stage III or Stage IV. In embodiments, the colorectal cancer is Stage I. In embodiments, the colorectal cancer is Stage II. In embodiments, the colorectal cancer is Stage III. In embodiments, the colorectal cancer is Stage IV. In embodiments, the early-onset colorectal cancer is Stage I or Stage II. In embodiments, the early-onset colorectal cancer is Stage III or Stage IV. In embodiments, the early-onset colorectal cancer is Stage I. In embodiments, the early-onset colorectal cancer is Stage II. In embodiments, the early-onset colorectal cancer is Stage III. In embodiments, the early-onset colorectal cancer is Stage IV. In embodiments, the late-onset colorectal cancer is Stage I or Stage II. In embodiments, the late-onset colorectal cancer is Stage III or Stage IV. In embodiments, the late-onset colorectal cancer is Stage I. In embodiments, the late-onset colorectal cancer is Stage II. In embodiments, the late-onset colorectal cancer is Stage III. In embodiments, the late-onset colorectal cancer is Stage IV.

In embodiments of the methods described herein, the patient is a human patient. In embodiments, the patient is less than 50 years old. In embodiments, the patient is less than 45 years old. In embodiments, the patient is less than 40 years old. In embodiments, the patient is ≤ 45 years old. In embodiments, the patient is ≤40 years old. In embodiments, the patient is ≥50 years old.

### Anti-Cancer Agents

In embodiments, the methods described herein comprise administering to a patient an effective amount of an anti-cancer agent. The anticancer treatment can be any drug known in the art as useful for treating colorectal cancer, such as chemotherapy, immunotherapy, or a combination thereof. In embodiments, the anti-cancer agent is a chemotherapeutic agent.

In embodiments, the chemotherapeutic agent is an alkylating agent, an antimetabolite compound, an anthracycline compound, an antitumor antibiotic, a platinum compound, a topoisomerase inhibitor, a vinca alkaloid, a taxane compound, an epothilone compound, or a combination of two or more thereof. In embodiments, the alkylating agent is carboplatin, chlorambucil, cyclophosphamide, melphalan, mechlorethamine, procarbazine, or thiotepa. In embodiments, the antimetabolite compound is azacitidine, capecitabine, cytarabine, gemcitabine, doxifluridine, hydroxyurea, methotrexate, pemetrexed, 6-thioguanine, 5-fluorouracil, or 6-mercaptopurine. In embodiments, the anthracycline compound is daunorubicin, doxorubicin, idarubicin, epirubicin, or mitoxantrone. In embodiments, the antitumor antibiotic is actinomycin, bleomycin, mitomycin, or valrubicin. In embodiments, the platinum compound is cisplatin or oxaliplatin. In embodiments, the topoisomerase inhibitor is irinotecan, topotecan, amsacrine, etoposide, teniposide, or eribulin. In embodiments, the vinca alkaloid is vincristine, vinblastine, vinorelbine, or vindesine. In embodiments, the taxane compound is paclitaxel or docetaxel. In embodiments, the epothilone compound is epothilone, ixabepilone, patupilone, or sagopilone. the chemotherapeutic agent comprises 5-fluorouracil, leucovorin, oxaliplatin, irinotecan, capecitabine, or a combination of two or more thereof.

In embodiments, the chemotherapeutic agent comprises 5-fluorouracil, leucovorin, oxaliplatin, irinotecan, capecitabine, or a combination of two or more thereof. In embodiments, the chemotherapeutic agent comprises everolimus, erlotinib, olaparib, mitomycin, sunitinib, gemcitabine, 5-fluorouracil, irinotecan, oxaliplatin, paclitaxel, capecitabine, cisplatin, docetaxel, or a combination of two or more thereof. In embodiments, the chemotherapeutic agent comprises 5-fluorouracil, oxaliplatin, irinotecan, capecitabine, or a combination of two or more thereof. In embodiments, the chemotherapeutic agent comprises gemcitabine, 5-fluorouracil, irinotecan, oxaliplatin, paclitaxel, capecitabine, cisplatin, docetaxel, or a combination of two or more thereof. In embodiments, the chemotherapeutic agent comprises gemcitabine. In embodiments, the chemotherapeutic agent comprises 5-fluorouracil. In embodiments, the chemotherapeutic agent comprises irinotecan. In embodiments, the chemotherapeutic agent comprises oxaliplatin. In embodiments, the chemotherapeutic agent comprises paclitaxel. In embodiments, the chemotherapeutic agent comprises capecitabine. In embodiments, the chemotherapeutic agent comprises cisplatin. In embodiments, the chemotherapeutic agent comprises docetaxel. In embodiments, the chemotherapeutic agent comprises further leucovorin.

“Chemotherapeutic” or “chemotherapeutic agent” is used in accordance with its plain ordinary meaning and refers to a chemical composition or compound having antineoplastic properties or the ability to inhibit the growth or proliferation of cells.

“Anti-cancer agent” is used in accordance with its plain ordinary meaning and refers to a composition (e.g. compound, drug, antagonist, inhibitor, modulator) having antineoplastic properties or the ability to inhibit the growth or proliferation of cells. In some embodiments, an anti-cancer agent is a chemotherapeutic. In embodiments, an anti-cancer agent is an agent identified herein having utility in methods of treating cancer. In embodiments, an anti-cancer agent is an agent approved by the FDA or similar regulatory agency of a country other than the USA, for treating cancer. Examples of anti-cancer agents include, but are not limited to, MEK (e.g. MEK1, MEK2, or MEK1 and MEK2) inhibitors (e.g. XL518, CI-1040, PD035901, selumetinib/AZD6244, GSK1120212/trametinib, GDC-0973, ARRY-162, ARRY-300, AZD8330, PD0325901, U0126, PD98059, TAK-733, PD318088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide, etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds (e.g. cisplatin, oxaloplatin, carboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002), mTOR inhibitors, antibodies (e.g., rituxan), 5-aza-2′-deoxycytidine, doxorubicin, vincristine, etoposide, gemcitabine, imatinib, geldanamycin, 17-N-allylamino-17-demethoxygeldanamycin (17-AAG), bortezomib, trastuzumab, anastrozole; angiogenesis inhibitors; antiandrogen, antiestrogen; antisense oligonucleotides; apoptosis gene modulators; apoptosis regulators; arginine deaminase; BCR/ABL antagonists; beta lactam derivatives; bFGF inhibitor; bicalutamide; camptothecin derivatives; casein kinase inhibitors (ICOS); clomifene analogues; cytarabine dacliximab; dexamethasone; estrogen agonists; estrogen antagonists; etanidazole; etoposide phosphate; exemestane; fadrozole; finasteride; fludarabine; fluorodaunorunicin hydrochloride; gadolinium texaphyrin; gallium nitrate; gelatinase inhibitors; gemcitabine; glutathione inhibitors; hepsulfam; immunostimulant peptides; insulin-like growth factor-1 receptor inhibitor; interferon agonists; interferons; interleukins; letrozole; leukemia inhibiting factor; leukocyte alpha interferon; leuprolide+estrogen+progesterone; leuprorelin; matrilysin inhibitors; matrix metalloproteinase inhibitors; MIF inhibitor; mifepristone; mismatched double stranded RNA; monoclonal antibody; mycobacterial cell wall extract; nitric oxide modulators; oxaliplatin; panomifene; pentrozole; phosphatase inhibitors; plasminogen activator inhibitor; platinum complex; platinum compounds; prednisone; proteasome inhibitors; protein A-based immune modulator; protein kinase C inhibitor; protein kinase C inhibitors, protein tyrosine phosphatase inhibitors; purine nucleoside phosphorylase inhibitors; ras farnesyl protein transferase inhibitors; ras inhibitors; ras-GAP inhibitor; ribozymes; signal transduction inhibitors; signal transduction modulators; single chain antigen-binding protein; stem cell inhibitor; stem-cell division inhibitors; stromelysin inhibitors; synthetic glycosaminoglycans; tamoxifen methiodide; telomerase inhibitors; thyroid stimulating hormone; translation inhibitors; tyrosine kinase inhibitors; urokinase receptor antagonists; steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guérin, levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to 111 In, 90Y, or 1311, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib, erlotinib, cetuximab, lapatinib, panitumumab, vandetanib, afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, or the like.

### Kits

Provided here are kits comprising components, such as reagents and reaction mixtures, to conduct the assays to detect the miRNA and mRNA as described herein. As part of the kit, materials and instruction are provided, e.g., for storage and use of kit components. In embodiments, the kits comprise one or more of the following: a RNA probe that can hybridize to a RNA biomarker, pairs of primers that under appropriate reaction conditions can prime amplification of at least a portion of a RNA marker or a RNA encoding a polypeptide marker (e.g., by PCR), instructions on how to use the kit, and a label or insert indicating regulatory approval for diagnostic or therapeutic use. In embodiments, the kit further includes RNA microarrays comprising RNA of the disclosure or molecules which specifically bind to the RNA described herein. In embodiments, standard techniques of microarray technology are utilized to assess expression of the RNA. Polynucleotide arrays, particularly arrays that bind RNA described herein, also can be used for diagnostic applications, such as for identifying subjects that have a condition characterized by expression of polypeptide biomarkers.

“Assaying” or “detecting” means using an analytic procedure to qualitatively assess or quantitatively measure the presence or amount or the functional activity of a target entity (e.g., miRNA, mRNA). For example, detecting the level of RNA (such as miRNA or mRNA) means using an analytic procedure (such as an in vitro procedure) to qualitatively assess or quantitatively measure the presence or amount of the RNA. In embodiments, raw expression values are normalized by performing quantile normalization relative to the reference distribution and subsequent log 10-transformation. In embodiments, when RNA expression is detected using the nCounter® Analysis System marketed by NanoString Technologies, the reference distribution is generated by pooling reported (i.e., raw) counts for the test sample and one or more control samples (preferably at least 2 samples, more preferably at least any of 4, 8 or 16 samples) after excluding values for technical (both positive and negative control) probes and without performing intermediate normalization relying on negative (background-adjusted) or positive (synthetic sequences spiked with known titrations).

The terms “probe” or “primer” refer to one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe or primers can be unlabeled or labeled as described below so that its binding to a target sequence can be detected (e.g., with a FRET donor or acceptor label). The probe or primer can be designed based on one or more particular (preselected) portions of a chromosome, e.g., one or more clones, an isolated whole chromosome or chromosome fragment, or a collection of polymerase chain reaction (PCR) amplification products. One of skill can adjust these factors to provide optimum hybridization and signal production for a given hybridization and detection procedures, and to provide the required resolution among different genes or genomic locations.

Probes and primers can also be immobilized on a solid surface (e.g., nitrocellulose, glass, quartz, fused silica slides), as in an array. Techniques for producing high density arrays can also be used for this purpose. One of skill will recognize that the precise sequence of particular probes and primers can be modified from the target sequence to a certain degree to produce probes that are “substantially identical” or “substantially complementary to” a target sequence, but retain the ability to specifically bind to (i.e., hybridize specifically to) the same targets from which they were derived.

The term “capable of hybridizing to” refers to a polynucleotide sequence that forms Watson-Crick bonds with a complementary sequence. One of skill will understand that the percent complementarity need not be 100% for hybridization to occur, depending on the length of the polynucleotides, length of the complementary region (e.g. 5, 10, 15, 20, 25, 30, 35, 40, 45, 50, 60, 70, 80, 90, 100, or more bases in length), and stringency of the conditions. For example, a polynucleotide (e.g., primer or probe) can be capable of binding to a polynucleotide having 60%, 65%, 70%, 75%, 80%, 85%, 90%, 91%, 92%, 93%, 94%, 95%, 96%, 97%, 98%, 99% or 100% complementarity over the stretch of the complementary region.

In embodiments, methods include detecting a level of a biomarker with a specific binding agent (e.g., an agent that binds to a protein or nucleic acid molecule). Exemplary binding agents include an antibody or a fragment thereof, a detectable protein or a fragment thereof, a nucleic acid molecule such as an oligonucleotide/polynucleotide comprising a sequence that is complementary to patient genomic DNA, miRNA or a cDNA produced from patient mRNA, or any combination thereof. In embodiments, an antibody is labeled with detectable moiety, e.g., a fluorescent compound, an enzyme or functional fragment thereof, or a radioactive agent. In embodiments, an antibody is detectably labeled by coupling it to a chemiluminescent compound. In embodiments, the presence of the chemiluminescent-tagged antibody is then determined by detecting the presence of luminescence that arises during the course of chemical reaction. Non-limiting examples of useful chemiluminescent labeling compounds are luminol, isoluminol, theromatic acridinium ester, imidazole, acridinium salt and oxalate ester.

In embodiments, a specific binding agent is an agent that has greater than 10-fold, preferably greater than 100-fold, and most preferably, greater than 1000-fold affinity for the target molecule as compared to another molecule. As the skilled artisan will appreciate the term specific is used to indicate that other biomarkers present in the sample do not significantly bind to the binding agent specific for the target molecule. In embodiments, the level of binding to a biomolecule other than the target biomarker results in a binding affinity which is at most only 10% or less, only 5% or less only 2% or less or only 1% or less of the affinity to the target molecule, respectively. A preferred specific binding agent will fulfill both the above minimum criteria for affinity as well as for specificity. For example, in embodiments an antibody has a binding affinity (e.g., Kd) in the low micromolar (10⁻⁶), nanomolar (10⁻⁷-10⁻⁹), with high affinity antibodies in the low nanomolar (10⁻⁹) or picomolar (10⁻¹²) range for its specific target biomarker.

In embodiments, the subject matter provides a composition comprising a binding agent, wherein the binding agent is attached to a solid support, (e.g., a strip, a polymer, a bead, a nanoparticle, a plate such as a multiwell plate, or an array such as a microarray). In embodiments relating to the use of a nucleic acid probe attached to a solid support (such as a microarray), a nucleic acid in a test sample may be amplified (e.g., using PCR) before or after the nucleic acid to be measured is hybridized with the probe. In embodiments, reverse transcription polymerase chain reaction (RT-PCR) is used to detect mRNA levels. In embodiments, a probe on a solid support is used, and miRNA (or a portion thereof) in a biological sample is converted to cDNA or partial cDNA and then the cDNA or partial cDNA is hybridized to a probe (e.g., on a microarray), hybridized to a probe and then amplified, or amplified and then hybridized to a probe. In embodiments, a strip may be a nucleic acid-probe coated porous or non-porous solid support strip comprising linking a nucleic acid probe to a carrier to prepare a conjugate and immobilizing the conjugate on a porous solid support. In embodiments, the support or carrier comprises glass, polystyrene, polypropylene, polyethylene, dextran, nylon, amylases, natural and modified celluloses, polyacrylamides, gabbros, and magnetite. In embodiments, the nature of the carrier can be either soluble to some extent or insoluble for the purposes of the present subject matter. In embodiments, the support material may have any structural configuration so long as the coupled molecule is capable of binding to a binding agent (e.g., an antibody). In embodiments, the support configuration may be spherical, as in a bead, or cylindrical, as in the inside surface of a test tube, or the external surface of a rod. In embodiments, the surface may be flat such as a plate (or a well within a multiwell plate), sheet, test strip, polystyrene beads. Those skilled in the art will know many other suitable carriers for binding antibody or antigen, or will be able to ascertain the same by use of routine experimentation.

In embodiments, a solid support comprises a polymer, to which an agent is chemically bound, immobilized, dispersed, or associated. In embodiments, a polymer support may be, e.g., a network of polymers, and may be prepared in bead form (e.g., by suspension polymerization). In embodiments, the location of active sites introduced into a polymer support depends on the type of polymer support. In embodiments, in a swollen-gel-bead polymer support the active sites are distributed uniformly throughout the beads, whereas in a macroporous-bead polymer support they are predominantly on the internal surfaces of the macropores. In embodiments, the solid support, e.g., a device, may contain a biomarker binding agent alone or together with a binding agent for at least one, two, three or more other biomarkers.

In embodiments, the cells in a biological sample are lysed to release a protein or nucleic acid. Numerous methods for lysing cells and assessing protein and nucleic acid levels are known in the art. In embodiments, cells are physically lysed, such as by mechanical disruption, liquid homogenization, high frequency sound waves, freeze/thaw cycles, with a detergent, or manual grinding. Non-limiting examples of detergents include Tween 20, Triton X-100, and sodium dodecyl sulfate (SDS). Non-limiting examples of assays for determining the level of a protein include HPLC, LC/MS, ELISA, immunoelectrophoresis, Western blot, immunohistochemistry, and radioimmunoassays. Non-limiting examples of assays for determining the level of an miRNA include Northern blotting, RT-PCR, RNA sequencing, and qRT-PCR.

In embodiments, once a suitable biological sample has been obtained, it is analyzed to quantitate the expression level of each of the biomarker genes. In embodiments, determining the expression level of a gene comprises detecting and quantifying RNA transcribed from that gene or a protein translated from such RNA. In embodiments, the RNA includes mRNA transcribed from the gene, and/or specific spliced variants thereof and/or fragments of such mRNA and spliced variants.

In embodiments, raw expression values are normalized by performing quantile normalization relative to the reference distribution and subsequent log 10-transformation. In embodiments, when the gene expression is detected using the nCounter® Analysis System marketed by NanoString® Technologies, the reference distribution is generated by pooling reported (i.e., raw) counts for the test sample and one or more control samples (preferably at least 2 samples, more preferably at least any of 4, 8 or 16 samples) after excluding values for technical (both positive and negative control) probes and without performing intermediate normalization relying on negative (background-adjusted) or positive (synthetic sequences spiked with known titrations). In embodiments, the T-effector signature score is then calculated as the arithmetic mean of normalized values for each of the genes in the gene signature.

A “detectable agent” or “detectable moiety” is a compound or composition detectable by appropriate means such as spectroscopic, photochemical, biochemical, immunochemical, chemical, magnetic resonance imaging, or other physical means. The RNA described herein and the expression level of the RNA described herein may be accomplished through the use of a detectable moiety in an assay or kit. A detectable moiety is a monovalent detectable agent or a detectable agent bound (e.g. covalently and directly or via a linking group) with another compound, e.g., a nucleic acid. Exemplary detectable agents/moieties for use in the present disclosure include an antibody ligand, a peptide, a nucleic acid, radioisotopes, paramagnetic metal ions, fluorophore, electron-dense reagents, enzymes (e.g., as commonly used in an ELISA), biotin, a biotin-avidin complex, a biotin-streptavidin complex, digoxigenin, magnetic beads (e.g., DYNABEADS® by ThermoFisher, encompassing functionalized magnetic beads such as DYNABEADS® M-270 amine by ThermoFisher), paramagnetic molecules, paramagnetic nanoparticles, ultrasmall superparamagnetic iron oxide nanoparticles, ultrasmall superparamagnetic iron oxide nanoparticle aggregates, superparamagnetic iron oxide nanoparticles, superparamagnetic iron oxide nanoparticle aggregates, monocrystalline iron oxide nanoparticles, monocrystalline iron oxide, nanoparticle contrast agents, liposomes or other delivery vehicles containing Gadolinium chelate molecules, gadolinium, radionuclides, fluorodeoxyglucose, any gamma ray emitting radionuclides, positron-emitting radionuclide, radiolabeled glucose, radiolabeled water, radiolabeled ammonia, biocolloids, microbubbles, iodinated contrast agents, barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide.

In embodiments, oligonucleotides in kits are capable of specifically hybridizing to a target region of a polynucleotide, such as for example, an RNA transcript or cDNA generated therefrom. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. The composition and length of each oligonucleotide in the kit will depend on the nature of the transcript containing the target region as well as the type of assay to be performed with the oligonucleotide and is readily determined by the skilled artisan.

In embodiments, the kit comprises reagents capable of detecting an expression level of RNA from a biological sample; wherein the RNA comprises miR-513a-5p, miR-628-3p, miR-193a-5p, miR-210, miR-4304, miR-194-3p, miR-4453, or a combination of two or more thereof. In embodiments, the kit comprises reagents capable of detecting an expression level of RNA from a tissue sample; wherein the RNA comprises miR-513a-5p, miR-628-3p, miR-193a-5p, miR-210, miR-4304, miR-194-3p, miR-4453, or a combination of two or more thereof. In embodiments, the kit comprises reagents capable of detecting an expression level of RNA from a tumor tissue sample; wherein the RNA comprises miR-513a-5p, miR-628-3p, miR-193a-5p, miR-210, miR-4304, miR-194-3p, miR-4453, or a combination of two or more thereof. In embodiments, the kit comprises reagents capable of detecting an expression level of RNA from a blood sample; wherein the RNA comprises miR-513a-5p, miR-628-3p, miR-193a-5p, miR-210, miR-4304, miR-194-3p, miR-4453, or a combination of two or more thereof. In embodiments, the kit comprises reagents capable of detecting an expression level of RNA from a blood sample; wherein the RNA comprises miR-513a-5p, miR-628-3p, miR-193a-5p, miR-210, or a combination of two or more thereof. In embodiments, the kit comprises reagents capable of detecting an expression level of RNA from a blood sample; wherein the RNA comprises miR-513a-5p, miR-628-3p, miR-193a-5p, and miR-210.

In embodiments, the disclosure provides a kit for detecting the RNA (e.g., miRNA, mRNA) described herein. In embodiments, the kit is an assay system including any one of assay reagents, assay controls, protocols, exemplary assay results, or combinations of these components designed to provide the user with means to evaluate the expression level of the RNA (e.g., miRNA, mRNA) described herein. In embodiments, the disclosure provides a kit for diagnosing colorectal cancer in a patent, including reagents for detecting RNA markers in a biological (e.g., blood) sample from a patient.

In embodiments, the kits comprise one or more of the following: a RNA probe that can hybridize to a RNA biomarker, pairs of primers that under appropriate reaction conditions can prime amplification of at least a portion of a RNA marker or a RNA encoding a polypeptide marker (e.g., by PCR), instructions on how to use the kit, and a label or insert indicating regulatory approval for diagnostic or therapeutic use. In embodiments, the kit further includes RNA microarrays comprising RNA of the disclosure or molecules which specifically bind to the RNA described herein. In embodiments, standard techniques of microarray technology are utilized to assess expression of the RNA. Polynucleotide arrays, particularly arrays that bind RNA described herein, also can be used for diagnostic applications, such as for identifying subjects that have a condition characterized by expression of polypeptide biomarkers, e.g., interstitial lung disease.

In addition, the means for detecting of the assay system can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide). The kit can also include suitable reagents for the detection of the reagent and/or for the labeling of positive or negative controls, wash solutions, dilution buffers and the like. The assay system can also include a set of written instructions for using the system and interpreting the results.

### Diagnosis

In embodiments of the methods described herein, the elevated level of gene expression is an elevated level of RNA (e.g., miRNA) expression. Levels of gene expression can be determined by methods known in the art, such as those described herein. In embodiments, the RNA is miRNA. In embodiments, RNA expression is detected by direct digital counting of nucleic acids, RNA sequencing (RNA-seq), quantitative reverse transcriptase polymerase chain reaction (RT-qPCR), quantitative polymerase chain reaction (qPCR), multiplex qPCR, microarray analysis, or a combination thereof. In embodiments, RNA expression is detected by RNA sequencing. RNA sequencing is a sequencing technique which uses next-generation sequencing (NGS) to reveal the presence and quantity of RNA in a biological sample. In embodiments, the gene expression level is an average of the gene expression level of the biomarker genes. In embodiments, the average of the gene expression level of the biomarker genes is an average of the normalized gene expression level of the biomarker genes. In embodiments, the gene expression level of the biomarker genes is a median of the gene expression level of the biomarker genes. In embodiments, the median of the gene expression level of the biomarker genes is a median of a normalized gene expression level of the biomarker genes. In embodiments, the gene expression level of the biomarker genes is the gene expression level of the biomarker genes normalized to a reference gene.

In embodiments, the individual elevated expression level of the RNA described herein are used to produce a colorectal cancer diagnosis; wherein the RNA comprises miR-513a-5p, miR-628-3p, miR-193a-5p, miR-210, miR-4304, miR-194-3p, miR-4453, or a combination of two or more thereof. In embodiments, the individual elevated expression level of the RNA described herein are used to produce a colorectal cancer diagnosis; wherein the RNA comprises miR-513a-5p, miR-628-3p, miR-193a-5p, miR-210, or a combination of two or more thereof. In embodiments, the elevated expression levels of the RNA are combined to form a risk score. In embodiments, the elevated expression levels of the RNA are weighted and combined to form a risk score.

Relative quantification relates the PCR signal of the target transcript in a treatment group to that of another sample such as the control (e.g., healthy individuals). The 2^{-ΔCt} method is a convenient way to analyze the relative changes in gene expression from real-time quantitative PCR experiments. The Ct (threshold cycle) method quantification was used for the evaluation of the expression level of each miRNA. The threshold cycle (Ct) is defined as the PCR cycle at which the fluorescent signal of the reporter dye crosses an arbitrarily placed threshold. This method allows to quantify the absolute expression of each miRNAs in each sample analyzed and then to calculate the different expression of each miRNA in sample versus the controls. These expression values of the RNA can be used individually to produce a risk score, can be added together to produce a risk score, or logistic regression analysis can be applied to produce a risk score based on weighted values of the expression levels of the RNA.

In embodiments, the disclosure provides methods of processing RNA expression data generated from the expression levels of the RNA in the biological sample obtained from a patient as described herein, for establishing the presence of a signature indicative of colorectal cancer, comprising the steps of (i) normalizing and/or scaling numeric values of the RNA expression data, (ii) refining the discriminatory power of individual RNA by statistically weighting some of the numeric values associated therewith, and (iii) summating the numeric values obtained from step (ii) to provide a composite expression score. In embodiments, the composite expression score obtained from step (iii) is compared to a control and the comparison allows the sample to be designated as positive or negative for colorectal cancer. In embodiments, the composite expression score is normalized. In embodiments, the composite expression score is scaled. In embodiments, the composite expression score is weighted. Weighted refers to the relevant value being adjusted to more appropriately reflect its contribution to the profile.

In embodiments, the risk score is determined using logistic regression analysis. In embodiments, the miRNA expression-based risk score is calculated as follows: Logit (P)=(−8.78655*miR-193a-5p)+(1.77237*miR-210)+(5.30584*miR-513a-5p)+(1.65455*miR-628-3p)+3.75375. In embodiments, the expression of level of each miRNA is calculated using 2^{-ΔCt} method, the normalized expression values are log₁₀ transformed, and then used in the equations herein.

### Embodiments 1-59

Embodiment 1. A method of treating colorectal cancer in a patient in need thereof, the method comprising administering to the patient an effective amount of an anti-cancer agent, administering to the patient an effective amount of radiation therapy, administering to the patient image-based screening, surgically removing all or a portion of the colon of the patient, or a combination of two or more thereof; wherein a biological sample obtained from the patient comprises an elevated expression level, relative to a control, of a RNA; wherein the RNA comprises miR-513a-5p, miR-628-3p, miR-193a-5p, miR-210, miR-4304, miR-194-3p, miR-4453, or a combination of two or more thereof.

Embodiment 2. A method of treating colorectal cancer in a patient in need thereof, the method comprising: (i) detecting an elevated expression level, relative to a control, of RNA in a biological sample obtained from the patient; wherein the RNA comprises miR-513a-5p, miR-628-3p, miR-193a-5p, miR-210, miR-4304, miR-194-3p, miR-4453, or a combination of two or more thereof, and (ii) administering to the patient an effective amount of an anti-cancer agent, administering to the patient an effective amount of radiation therapy, administering to the patient image-based screening, surgically removing all or a portion of the colon of the patient, or a combination of two or more thereof.

Embodiment 3. The method of embodiment 1 or 2, comprising administering to the patient an effective amount of an anti-cancer agent.

Embodiment 4. The method of embodiment 1 or 2, comprising surgically removing all or a portion of the colon of the patient.

Embodiment 5. The method of embodiment 1 or 2, comprising administering to the patient an effective amount of an anti-cancer agent and surgically removing all or a portion of the colon of the patient.

Embodiment 6. The method of embodiment 1 or 2, comprising administering to the patient an effective amount of an anti-cancer agent, administering to the patient an effective amount of radiation therapy, and surgically removing all or a portion of the colon of the patient.

Embodiment 7. The method of embodiment 1 or 2, comprising administering to the patient an effective amount of an anti-cancer agent and administering to the patient an effective amount of radiation therapy.

Embodiment 8. A method of diagnosing a patient with colorectal cancer, the method comprising: (i) detecting the expression level of RNA in a biological sample obtained from the patient, and (ii) diagnosing the patient as having colorectal cancer when the biological sample has an elevated expression level, relative to a control, of the RNA; wherein the RNA comprises miR-513a-5p, miR-628-3p, miR-193a-5p, miR-210, miR-4304, miR-194-3p, miR-4453, or a combination of two or more thereof.

Embodiment 9. A method of monitoring treatment in a patient having colorectal cancer or monitoring risk for developing colorectal cancer in a patient, the method comprising: (i) detecting the expression level of RNA in a biological sample obtained from the patient at a first time point, (ii) detecting the expression level of the RNA in a biological sample obtained from the patient at a second time point, wherein the second time point is later than the first time point, and (iii) comparing the expression level of the RNA at the second time point to the expression level of the RNA at the first time point, thereby monitoring treatment or monitoring risk; wherein the RNA comprises miR-513a-5p, miR-628-3p, miR-193a-5p, miR-210, miR-4304, miR-194-3p, miR-4453, or a combination of two or more thereof.

Embodiment 10. The method of embodiment 9, comprising monitoring risk for developing colorectal cancer in the patient.

Embodiment 11. The method of embodiment 10, wherein an elevated expression level of RNA at the second point in time when compared to the expression level of RNA at the first point in time indicates that the patient has an increased risk of developing colorectal cancer.

Embodiment 12. The method of embodiment 9, comprising monitoring treatment in a patient having colorectal cancer.

Embodiment 13. The method of embodiment 12, further comprising surgically removing all or a portion of the colon of the patient after step (i) and before step (ii).

Embodiment 14. The method of embodiment 12, further comprising administering to the patient an effective amount of an anti-cancer agent after step (i) and before step (ii).

Embodiment 15. The method of embodiment 12, further comprising surgically removing all or a portion of the colon of the patient and administering to the patient an effective amount of an anti-cancer agent after step (i) and before step (ii).

Embodiment 16. A method of detecting RNA in a patient with colorectal cancer, the method comprising detecting an elevated expression level, relative to a control, of RNA in a biological sample obtained from the patient; wherein the RNA comprises miR-513a-5p, miR-628-3p, miR-193a-5p, miR-210, miR-4304, miR-194-3p, miR-4453, or a combination of two or more thereof.

Embodiment 17. The method of any one of embodiments 8 to 16, further comprising administering to the patient an effective amount of an anti-cancer agent, administering to the patient an effective amount of radiation therapy, administering to the patient image-based screening, surgically removing all or a portion of the colon of the patient, or a combination of two or more thereof.

Embodiment 18. The method of any one of embodiments 1 to 17, wherein the RNA comprises miR-513a-5p.

Embodiment 19. The method of any one of embodiments 1 to 18, wherein the RNA comprises miR-210.

Embodiment 20. The method of any one of embodiments 1 to 19, wherein the RNA comprises miR-628-3p.

Embodiment 21. The method of any one of embodiments 1 to 20, wherein the RNA comprises miR-193a-5p.

Embodiment 22. The method of any one of embodiments 1 to 21, wherein the RNA comprises miR-4304.

Embodiment 23. The method of any one of embodiments 1 to 22, wherein the RNA comprises miR-194-3p.

Embodiment 24. The method of any one of embodiments 1 to 23, wherein the RNA comprises miR-4453.

Embodiment 25. The method of any one of embodiments 1 to 17, wherein the RNA comprises miR-513a-5p, miR-628-3p, miR-193a-5p, miR-210, or a combination of two or more thereof.

Embodiment 26. The method of any one of embodiments 1 to 17, wherein the RNA comprises at least two of miR-513a-5p, miR-628-3p, miR-193a-5p, and miR-210.

Embodiment 27. The method of any one of embodiments 1 to 17, wherein the RNA comprises at least three of miR-513a-5p, miR-628-3p, miR-193a-5p, and miR-210.

Embodiment 28. The method of any one of embodiments 1 to 17, wherein the RNA comprises miR-513a-5p, miR-628-3p, miR-193a-5p, and miR-210.

Embodiment 29. The method of any one of embodiments 1 to 17, wherein the RNA consists of miR-513a-5p, miR-628-3p, miR-193a-5p, and miR-210.

Embodiment 30. The method of any one of embodiments 1 to 17, wherein the RNA comprises at least two of miR-513a-5p, miR-628-3p, miR-193a-5p, miR-210, miR-4304, miR-194-3p, and miR-4453.

Embodiment 31. The method of any one of embodiments 1 to 17, wherein the RNA comprises at least three of miR-513a-5p, miR-628-3p, miR-193a-5p, miR-210, miR-4304, miR-194-3p, and miR-4453.

Embodiment 32. The method of any one of embodiments 1 to 17, wherein the RNA comprises at least four of miR-513a-5p, miR-628-3p, miR-193a-5p, miR-210, miR-4304, miR-194-3p, and miR-4453.

Embodiment 33. The method of any one of embodiments 1 to 17, wherein the RNA comprises at least five of miR-513a-5p, miR-628-3p, miR-193a-5p, miR-210, miR-4304, miR-194-3p, and miR-4453.

Embodiment 34. The method of any one of embodiments 1 to 17, wherein the RNA comprises at least six of miR-513a-5p, miR-628-3p, miR-193a-5p, miR-210, miR-4304, miR-194-3p, and miR-4453.

Embodiment 35. The method of any one of embodiments 1 to 17, wherein the RNA comprises miR-513a-5p, miR-628-3p, miR-193a-5p, miR-210, miR-4304, miR-194-3p, and miR-4453.

Embodiment 36. The method of any one of embodiments 1 to 17, wherein the RNA consists of miR-513a-5p, miR-628-3p, miR-193a-5p, miR-210, miR-4304, miR-194-3p, and miR-4453.

Embodiment 37. The method of any one of embodiments 1 to 36, wherein the colorectal cancer is early-onset colorectal cancer.

Embodiment 38. The method of any one of embodiments 1 to 36, wherein the colorectal cancer is late-onset colorectal cancer.

Embodiment 39. The method of any one of embodiments 1 to 38, wherein the colorectal cancer is Stage I or Stage II.

Embodiment 40. The method of any one of embodiments 1 to 38, wherein the colorectal cancer is Stage III or Stage IV.

Embodiment 41. The method of any one of embodiments 1 to 40, wherein the biological sample is a blood sample.

Embodiment 42. The method of embodiment 41, wherein the blood sample is a serum sample.

Embodiment 43. The method of embodiment 41, wherein the blood sample is a plasma sample.

Embodiment 44. The method of any one of embodiments 1 to 40, wherein the biological sample is a tissue sample.

Embodiment 45. The method of any one of embodiments 1 to 44, wherein the patient is a human patient.

Embodiment 46. The method of any one of embodiments 1 to 45, wherein the patient is less than 50 years old.

Embodiment 47. The method of embodiment 46, wherein the patient is less than 45 years old.

Embodiment 48. The method of any one of embodiments 1 to 45, wherein the patient ≥50 years old.

Embodiment 49. The method of any one of embodiments 1-3, 5-7, 14, 15, and 17-48, wherein the anti-cancer agent is a chemotherapeutic agent.

Embodiment 50. The method of embodiment 49, wherein the chemotherapeutic agent comprises 5-fluorouracil, leucovorin, oxaliplatin, irinotecan, capecitabine, or a combination of two or more thereof.

Embodiment 51. The method of embodiment 49, wherein the chemotherapeutic agent is an alkylating agent, an antimetabolite compound, an anthracycline compound, an antitumor antibiotic, a platinum compound, a topoisomerase inhibitor, a vinca alkaloid, a taxane compound, an epothilone compound, or a combination of two or more thereof.

Embodiment 52. The method of embodiment 51, wherein the alkylating agent is carboplatin, chlorambucil, cyclophosphamide, melphalan, mechlorethamine, procarbazine, or thiotepa; the antimetabolite compound is azacitidine, capecitabine, cytarabine, gemcitabine, doxifluridine, hydroxyurea, methotrexate, pemetrexed, 6-thioguanine, 5-fluorouracil, or 6-mercaptopurine; the anthracycline compound is daunorubicin, doxorubicin, idarubicin, epirubicin, or mitoxantrone; the antitumor antibiotic is actinomycin, bleomycin, mitomycin, or valrubicin; the platinum compound is cisplatin or oxaliplatin; the topoisomerase inhibitor is irinotecan, topotecan, amsacrine, etoposide, teniposide, or eribulin; the vinca alkaloid is vincristine, vinblastine, vinorelbine, or vindesine; the taxane compound is paclitaxel or docetaxel; and the epothilone compound is epothilone, ixabepilone, patupilone, or sagopilone.

Embodiment 53. The method of any one of embodiments 1-8 and 16-52, wherein the control is a patient or population of patients that do not have cancer.

Embodiment 54. The method of any one of embodiments 1-8 and 16-52, wherein the control is a patient or population of patients that do not have colorectal cancer.

Embodiment 55. A kit comprising reagents capable of detecting an expression level of RNA from a biological sample; wherein the RNA comprises miR-513a-5p, miR-628-3p, miR-193a-5p, miR-210, miR-4304, miR-194-3p, miR-4453, or a combination of two or more thereof.

Embodiment 56. The kit of embodiment 55, wherein the RNA comprises miR-513a-5p, miR-628-3p, miR-193a-5p, miR-210, or a combination of two or more thereof.

Embodiment 57. The kit of embodiment 55 or 56, wherein the biological sample is a blood sample.

Embodiment 58. The kit of embodiment 57, wherein the blood sample is a serum sample or a plasma sample.

Embodiment 59. The kit of embodiment 55 or 56, wherein the biological sample is a tissue sample.

### EXAMPLE

### Summary of Example 1

Background and Aims: Early-onset colorectal cancer (EOCRC) is a distinct clinical and molecular entity with poor survival outcomes compared to late onset CRC (LOCRC). Although the incidence of EOCRC is rising, current CRC screening strategies have several limitations in diagnostic performance for EOCRC. In view of this clinical challenge, novel and robust biomarkers for detection of EOCRC are necessary. The aim of this study is to develop a circulating miRNA signature for the diagnosis of patients with EOCRC. Methods: A systematic discovery approach by analyzing a large, publicly available, noncoding RNA expression profiling dataset (GSE115513) was used. A panel of miRNAs was identified, which was subsequently validated in blood samples from EOCRC patients in two independent cohorts (n=149) compared with controls (n=110) and pre/post-operative plasma specimens (n=22) using qRT-PCR assays. Results: In the discovery phase, four miRNAs were found to be expressed in blood samples. A combination signature of these four miRNAs (miR-193a-5p, miR-210, miR-513a-5p and miR-628-3p) yielded an area under the curve (AUC) of 0.92 (95% CI=0.85-0.96) for identification of EOCRC in the training cohort. The miRNA panel performance was then confirmed in an independent validation cohort (AUC=0.88, 95% CI=0.82-0.93). Moreover, the miRNA panel robustly identified early stage EOCRC patients (P<0.001). The decreased expression of miRNAs in post-surgery plasma specimens indicated their tumor-specificity. Conclusions: Our novel miRNA signature for the diagnosis of EOCRC has the potential to identify EOCRC patients with high accuracy for clinical application in the non-invasive diagnosis of EOCRC.

### Example 1

Liquid biopsies have become highly topical in the field of diagnosis for various malignancies, and are gaining attention for overcoming some of the limitations of current screening strategies. In particular, blood remains the most attractive substrate for developing liquid biopsy-based assays as it is widely accepted by patients, and carries cancer-derived cargo released into the systemic circulation including proteins, metabolites and nucleic acids. In this context, work from our group and others has previously shown that circulating microRNAs (miRNAs) reflect physiological and pathological alterations in patients with CRC, and have the potential to serve as important surrogates as minimally invasive diagnostic biomarkers. miRNAs are short, 18-24-nucleotide, non-coding RNAs that play a pivotal role in gene regulation. Since miRNA expression is generally stable in tissues, blood, stool, and other bodily fluids, they have emerged as promising candidates for developing liquid biopsy biomarkers in human cancers. While, miRNAs have been previously explored in diagnosis of LOCRC (also known as CRC), to the best of our knowledge, no systematic studies have yet focused on identifying such biomarkers for the detection of patients with EOCRC.

Given the rise in incidence of EOCRC and the challenges for its early detection, the inventors performed a systematic, genome-wide analysis to comprehensively identify a miRNA signature for the early detection of patients with EOCRC. Following the biomarker discovery, this signature was rigorously validated in multiple independent cohorts of patients, and finally established a novel liquid biopsy assay for the detection of patients with early-stage EOCRC.

### Materials and Methods

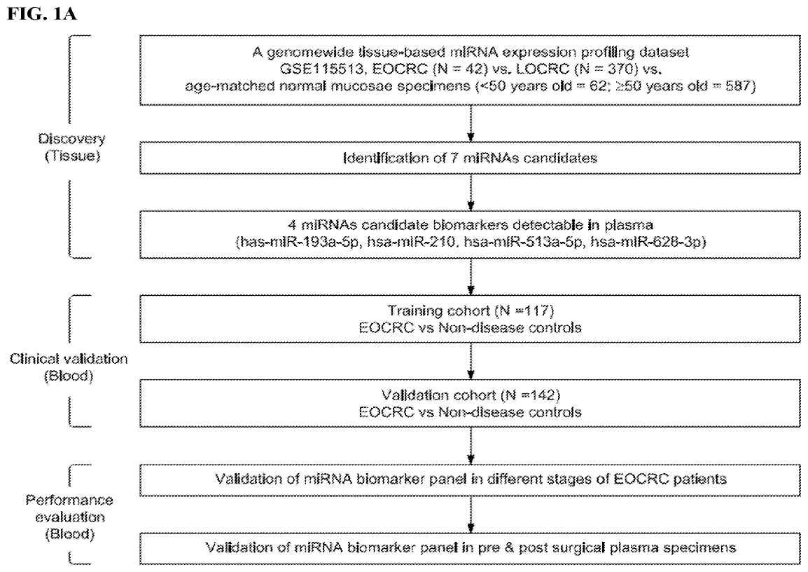

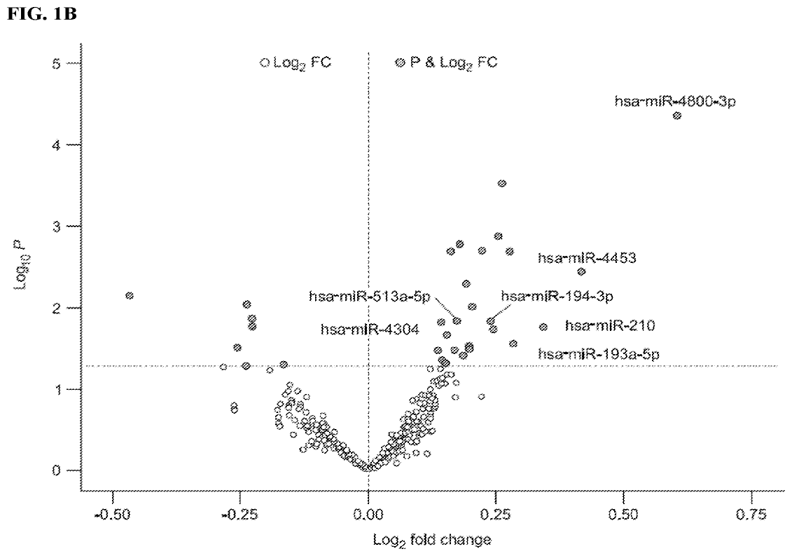

The present study employed a two-phase design. In the initial biomarker discovery phase, transcriptomic data from miRNA expression profiling dataset was systematically analyzed for the identification of a clinically translatable miRNAs that detects EOCRC. In the second phase, plasma specimens from multiple clinical cohorts were used to validate the performance of biomarkers selected in the discovery phase. In this clinical validation phase, quantitative reverse-transcription PCR (qRT-PCR) assays were employed to evaluate and compare the expression levels of candidate miRNAs in plasma specimens from individuals with EOCRC and those of no disease. The overall workflow for this study is illustrated in FIG. 1A.

Identification and Selection of Candidate miRNAs

A large, publicly available, miRNA expression profiling dataset (GSE115513, n=1061) was analyzed for the systematic discovery of miRNA biomarkers specific for patients with EOCRC. The normalized and pre-processed miRNA expression data was downloaded from the Gene Expression Omnibus (GEO). The miRNA expression data from paraffin-embedded tissues with patients with either stage I/II EOCRC (n=42) or LOCRC (n=370), as well as age-matched normal mucosa samples (<50 years old=62; ≥50 years old=587), respectively, were analyzed to identify candidate miRNAs, preferentially those that were upregulated in EOCRC. The differential expression analysis was performed using limma (version 3.38.3). Furthermore, the effect of any inadvertent potential bias that might exist due to age was mitigated by removing the miRNAs with a significant (P<0.05) change in the expression in the normal samples stratified by age (<50 and >50 years of age). A multivariate logistic regression model was built to establish the diagnostic potential of upregulated miRNAs in patients with EOCRC. Multi-collinearity was evaluated by calculating a variance inflation factor (VIF) for each predictor. The VIF values were calculated for each parameter in the model, and if found higher than the cutoff (=2), sequentially the predictor with the highest VIF was dropped, recalculated, and repeated until all values were below the cutoff.

### Patient Cohorts and Specimens

For this study, a total of 349 plasma samples, which included 117 samples in the training cohort (72 from patients with EOCRC and 45 from healthy donors <50 years old or non-disease controls) from 3 Japanese hospitals (National Cancer Center Hospital, Mie University, and Nagoya University), and 142 samples in the validation cohort (77 EOCRC and 65 non-disease controls) from Spanish hospitals (Hospital Universitario de Canarias, Hospital Clinic de Barcelona, Hospital Universitario de Donostia and Hospital Universitario Fundación Jiménez Díaz) were evaluated. For additional analyses, 44 specimens from patients with LOCRCs and 24 age-matched non-disease controls (≥50 years old), as well as a subset of 22 plasma specimens from patients that were obtained both pre- and post-operatively were used. Tumor staging was performed according to the Sixth Edition of the American Joint Commission on Cancer TNM staging system. The study was conducted in accordance with Declaration of Helsinki. Written informed consent was obtained from all patients, and the study was approved by the institutional review board of all participating institutions.

RNA extraction, cDNA synthesis and quantification of miRNAs using qRT-PCR

Total RNA was extracted from plasma samples using miRNeasy Serum/Plasma Kits (Qiagen, Valencia, CA; catalog number 217184) following manufacturer's protocol. Briefly, 250 μL of plasma was thawed on ice and centrifuged at 16,000×g at 4° C. for 10 minutes to remove cellular debris. Thereafter, 200 μL of supernatant was lysed in 1000 μL of QIAzol Lysis Reagent. After incubation for 5 minutes, 25 fmol of synthetic cel-miR-39 (Syn-cel-miR-39-3p miScript miRNA Mimic, Qiagen; catalog number MSY0000010) was added to each sample as an external spiked-in control. Total RNA, including small RNA, was extracted and eluted in 30 μL of RNase-free water using a QIAcube (Qiagen). Complementary DNA (cDNA) from total RNA was synthesized following miRCURY LNA (Qiagen). First-Strand cDNA Synthesis protocol was employed with slight modifications. qRT-PCR analysis was performed using the SensiFAST™ probe Lo-ROX Kit (Bioline, London, UK) on the QuantStudio 7 Flex Real Time PCR System (Applied Biosystems, Foster City, CA), and miRNA expression levels were evaluated with Applied Biosystems QuantStudio 7 Flex Real Time PCR System Software. The relative abundance of target transcripts was evaluated and normalized against the control using the 2^{-ΔΔCt} method. After the evaluation of different endogenous control miRNAs including U6, miR-16-5p and miR-103a-3p, miR-103a-3p was determined to be the optimal control based on expression and uniform threshold cycle (Ct) values across all samples (non-disease controls and cancer). Normalized values were further log₁₀ transformed. (Refs 37, 38)

### Statistical Analysis

The selection criteria to identify differentially expressed miRNAs included a log₂ fold change higher than zero and a p-value of less than or equal to 0.05. The data pre-processing and handling was performed using R/Bioconductor. Multicollinearity was tested using vif function in “rms”, and R library. A generalized multivariate regression model was built. Receiver operator characteristic (ROC) curves, areas under curve (AUC), positive predictive values (PPV), and negative predictive values (NPV) were determined to measure the performance of the diagnostic model. Thereafter, the Youden's index derived optimal cut-off thresholds were used to divide the training cohort patients with low vs. high-risk scores for diagnosing EOCRC. In order to analyze the miRNA model performance in different stages, we divided our cohort in stage I/II vs. III-IV EOCRC and analyzed its diagnostic performance. Statistical analyses for this study were performed using GraphPad Prism version 8.0, Medcalc statistical software V.16.2.0 (Medcalc Software bvba, Ostend, Belgium), and R (3.5.0, R Development Core Team, https://cran.r-project.org/). All p-values less than 0.05 were considered significant.

### Results

Genome-Wide Transcriptomic Profiling Identifies a 7-miRNA Tissue-Based Signature for the Detection of Patients with EOCRC

The first objective of this project was to identify a systematic and comprehensive miRNA signature from genome-wide transcriptomic profiling data available for patients with early-stage EOCRC compared to those with LOCRC and age-matched controls. In these analyses, 28 miRNAs were significantly up-regulated in patients with stage I-II EOCRC tissue samples compared to the normal mucosae (P<0.05). To enhance the overall specificity of the discovered markers, all miRNAs that overlapped between EOCRC and LOCRC patients were removed to increase the diagnostic potential of the markers that preferentially allowed identification of patients with EOCRC. Such a bioinformatic prioritization effort resulted in a panel of 11 miRNAs that were significantly upregulated in patients with EOCRC (P<0.05). Subsequently, after removing the biomarkers that exhibited collinearity with each other and did not offer any additional diagnostic contribution to the marker panel, a final panel of seven miRNAs that represented up-regulated markers in EOCRC patients (FIG. 1B) was prioritized. Finally a multivariate logistic regression model established the association of these seven miRNAs (hsa-miR-4304, hsa-miR-513a-5p, hsa-miR-628-3p, hsa-miR-194-3p, hsa-miR-193a-5p, hsa-miR-210 and hsa-miR-4453) in patients with stage I/II EOCRC, which exhibited a diagnostic AUC value of 0.82 (95% CI=0.73-0.91, P<0.001), with a corresponding a sensitivity of 0.72 and specificity of 0.84 for the detection of patients with early stage EOCRC (FIG. 1C). The data highlighted that this biomarker panel possessed remarkable diagnostic potential for the diagnosis of patients with EOCRC.

Establishment and Validation of Diagnostic Performance of a Circulating miRNA Panel in Independent Cohorts of Patients with EOCRC

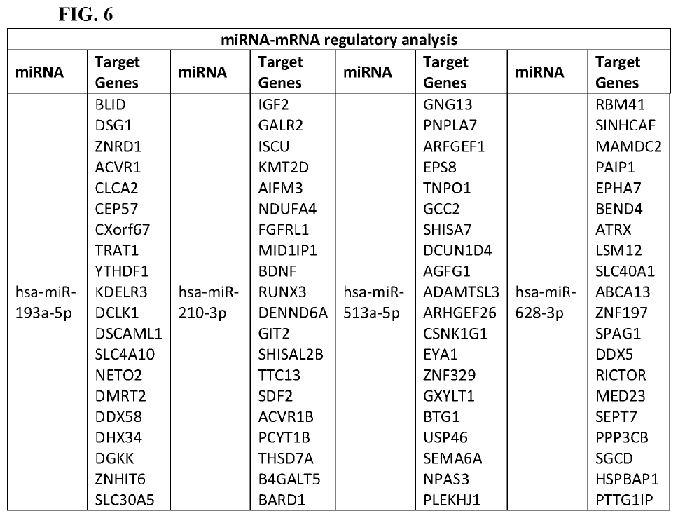

Since the primary objective was to develop a minimally invasive, liquid biopsy based assay for the identification of patients with EOCRC in clinical settings, the validation of diagnostic performance of this miRNA panel in actual clinical settings was warranted. In order to develop a blood-based assay, the tissue-based markers identified during the biomarker discovery phase were tested for detectability in plasma samples. Four of the seven tissue-based markers (hsa-miR-513a-5p, hsa-miR-628-3p, hsa-miR-193a-5p and hsa-miR-210) were detectable in these plasma samples. The corresponding genes for these differentially expressed miRNA are shown in FIG. 6.

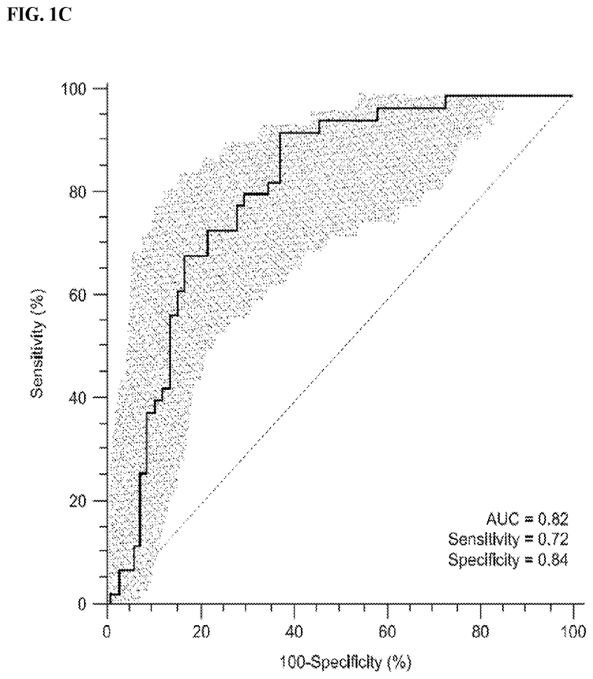

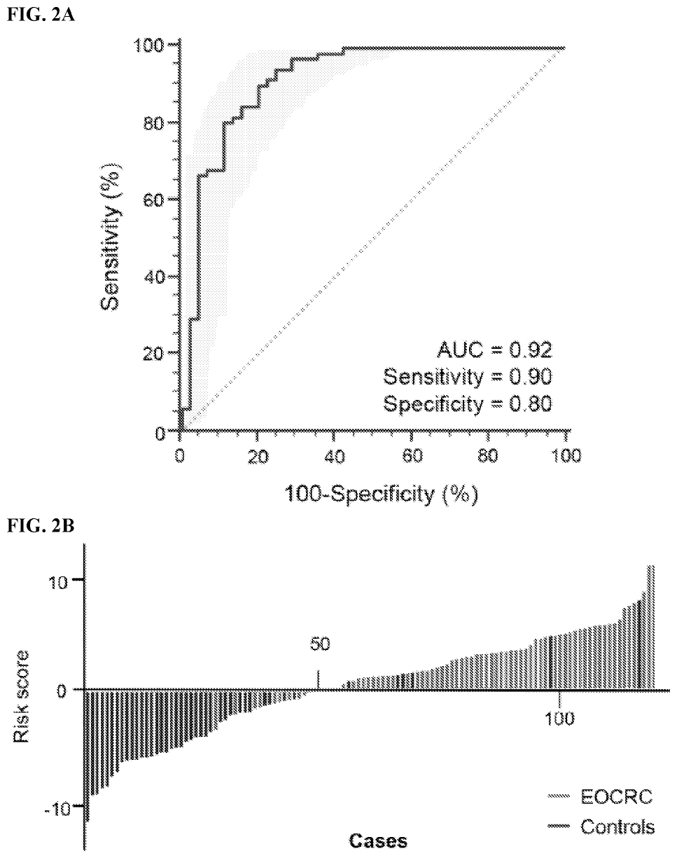

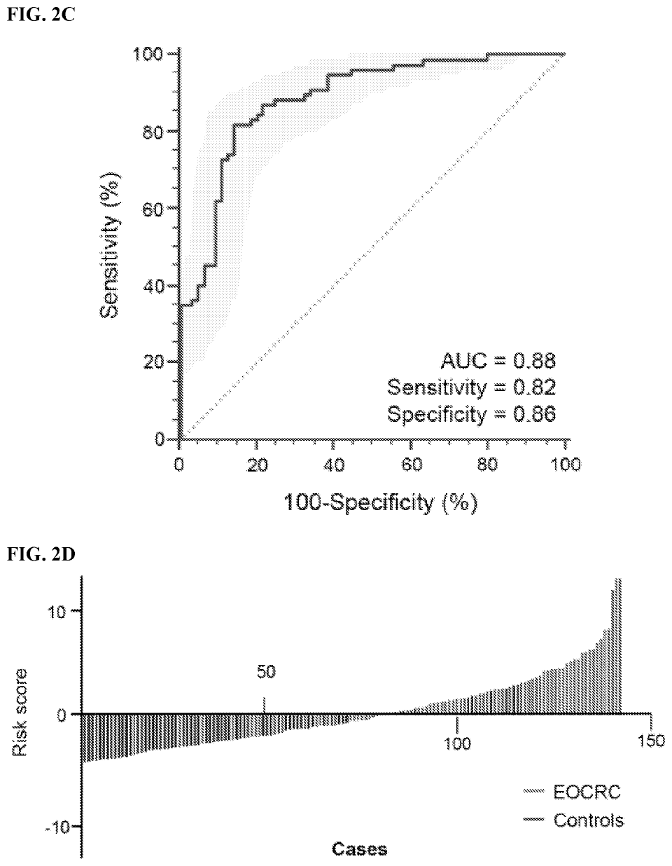

Subsequently, to establish the diagnostic potential of this miRNA panel for EOCRC patients, its performance in blood specimens obtained from two large, independent clinical cohorts was examined by using quantitative qRT-PCR assays. The clinical features of the patient cohort are summarized in Table 1 (clinicopathologic characteristics of the training and validation cohorts). In these experiments, using logistic regression analysis, the performance of this biomarker panel was trained in a cohort of patients from Japan [n=117 (72 EOCRC and 45 non-disease controls)]. The miRNA expression-based risk scores for each patient with EOCRC were calculated as follows: Logit (P)=(−8.78655*MIR193)+(1.77237*MIR210)+(5.30584*MIR513)+(1.65455*MIR628)+3.75375. This logistic regression based four miRNA risk-assessment model demonstrated an excellent diagnostic performance in blood specimens of patients with EOCRC with an AUC value of 0.92 (95% CI=0.85-0.96, P<0.001; FIG. 2A), with a corresponding sensitivity of 0.90, specificity of 0.80, PPV of 0.88 and NPV of 0.84 (Table 2-summary of diagnostic performance of miRNA-based biomarker panel in the training and validation cohorts).

TABLE 1

| | Training cohort | | Validation cohort | |
|---|---|---|---|---|
| | (N = 117) | | (N = 142) | |
| | EOCRC | Controls | EOCRC | Controls |
| | (N = 72) | (N = 45) | (N = 77) | (N = 65) |
| Age (median, | 44 (21-49) | 37 (26-49) | 43 (24-49) | 45 (39-49) |
| range) | | | | |
| Gender (n, %) | | | | |
| Male | 32 (44.4%) | 27 (60.0%) | 37 (48.1%) | 33 (50.8%) |
| Female | 40 (55.6%) | 18 (40.0%) | 40 (51.9%) | 32 (49.2%) |
| Localization | | | | |
| Right side | 19 (26.4%) | — | 17 (22.1%) | — |
| Left side | 36 (50.0%) | — | 32 (41.6%) | — |
| Rectum | 17 (23.6%) | — | 28 (22.1%) | — |
| Stage (TNM) | | | | |
| I | 20 (27.8%) | — | 9 (11.7%) | — |
| II | 19 (26.4%) | — | 19 (24.7%) | — |
| III | 25 (34.7%) | — | 29 (37.7%) | — |
| IV | 8 (11.1%) | — | 20 (26.0%) | — |

TABLE 2

| | Training cohort | Validation cohort |
|---|---|---|
| | (%, CI) | (%, CI) |
| Area Under the Curve (AUC) | 0.92 (0.85-0.96) | 0.88 (0.82-0.93) |
| Accuracy | 0.86 (0.79-0.92) | 0.84 (0.77-0.89) |
| Sensitivity | 0.90 (0.81-0.96) | 0.82 (0.71-0.90) |
| Specificity | 0.80 (0.65-0.90) | 0.86 (0.75-0.94) |
| Positive Predictive Value (PPV) | 0.88 (0.78-0.94) | 0.88 (0.78-0.94) |
| Negative Predicative Value (NPV) | 0.84 (0.69-0.93) | 0.80 (0.69-0.89) |

In view of the encouraging results of the blood-based panel for the detection of EOCRC in the training cohort, the robustness and accuracy of our risk-assessment model were assessed in another large independent validation cohort from Spain [n=142 (77 EOCRC and 65 non-disease controls)]. These validation efforts confirmed the earlier findings and yielded an AUC value of 0.88 (95% CI=0.82-0.93, P<0.001; FIG. 2B) with a sensitivity of 0.82, a specificity of 0.86, PPV of 0.88 and NPV of 0.80. Taken together, the genome-wide transcriptomic profiling approach was indeed robust, as it identified the biomarkers that were successfully trained and validated in plasma specimens from independent cohorts of patients with EOCRC, hence highlighting their translational potential in the clinic for the detection of this malignancy in early stages.

The Circulating miRNA Panel Identifies Early Stage (Stage I and II) and Late Stage (Stage III and IV) EOCRC Patients

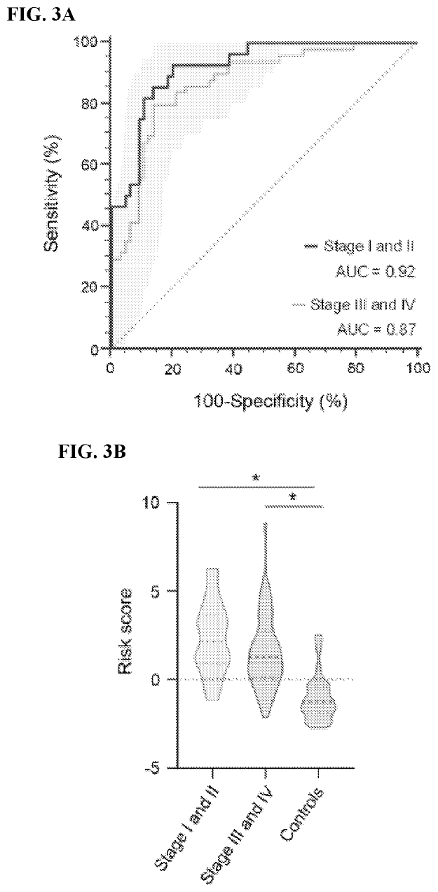

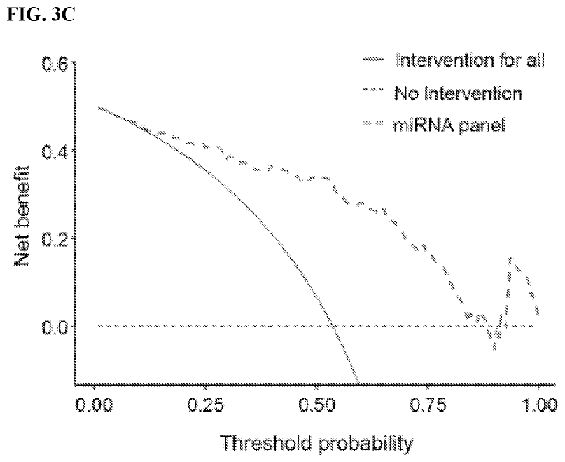

One approach for improving the survival outcomes in patients with EOCRC is to attain an earlier diagnosis. Therefore, whether this biomarker panel performed better in subgroups of EOCRC patients based upon their tumor stage, i.e., early stage (Stage I and II) vs late stage (Stage III and IV) was questioned. It was exciting to observe that, in the validation cohort, this circulating biomarker panel revealed excellent diagnostic performance in the identification of early-stage cancers (stage I/II) with an AUC of 0.92 (95% CI 0.84-0.96, sensitivity 0.92, specificity 0.80). On the other hand, regarding late-stage cancers (stage III/IV), the miRNA panel showed relatively fair performance as well, with an AUC of 0.87 (95% CI 0.79-0.92, sensitivity 0.79, specificity 0.86; FIG. 3A). Furthermore, the distribution of risk scores in early stage vs. late stage EOCRC patients relative to the non-disease controls was evaluated. It was quite reassuring to observe that this circulating biomarker panel successfully identified patients with both early and late stage EOCRCs in the validation cohort (P<0.001, respectively; FIG. 3B). Collectively, these results highlight that this four-miRNA circulating biomarker panel is robust in the identification of patients with early disease stages and late disease stages. These data are significant since early detection is essential to improve the survival outcomes in patients with EOCRC.

The miRNA Panel Offers a Significant Benefit Compared to Current Screening Approaches

The clinical usefulness of screening strategies should be estimated by the trade-off between the harm and diagnosis. Since the diagnosis of CRC patients is achieved by a screening test through colonoscopy and an invasive biopsy, false positive or false negative cases would be detrimental to individuals undergoing such screening. To estimate the clinical significance of the miRNA panel, decision curve analysis (DCA) was performed (FIG. 3C). The DCA curve revealed that the miRNA panel achieved a higher net benefit regardless of threshold probability in comparison to intervention for all patients or none of the patients. These findings indicate that this miRNA panel will offer clinical benefit with regards to the avoidance of physical harm and misdiagnosis.

### The Expression Levels of Circulating MiRNAs Significantly Decrease Following Surgical Removal of the CRC

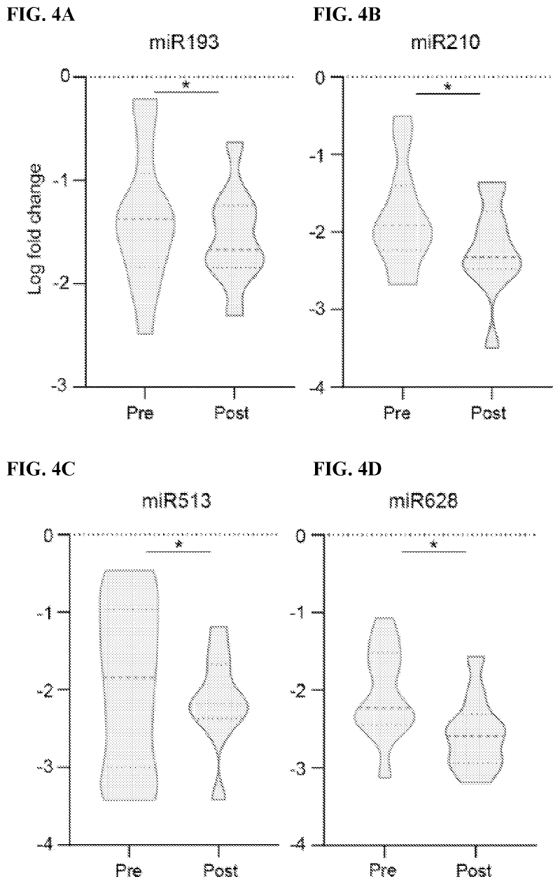

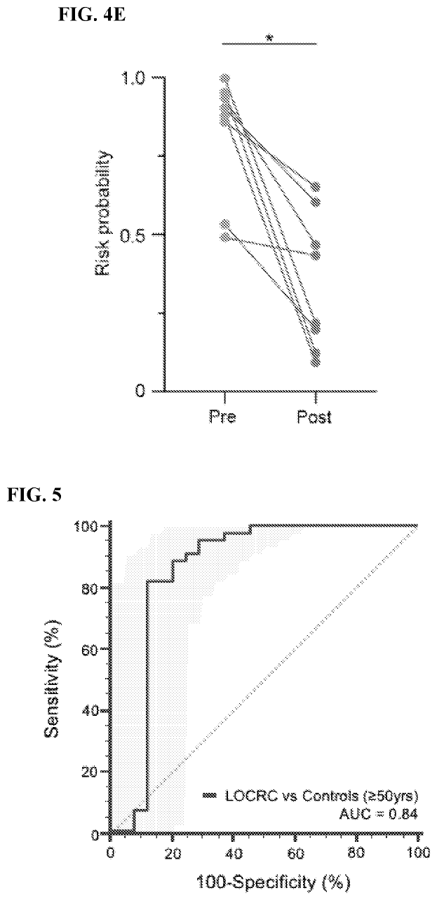

The hypothesis behind the expression of circulating biomarkers is that tumor cells constantly shed cellular cargo into the systemic circulation, which is amenable to detection in a liquid biopsy. Presuming that the expression of these biomarkers diminishes once their source (i.e., the cancer) is resected, the expression of the miRNA biomarker panel was analyzed in a subset of EOCRC patients where the baseline plasma specimens were collected prior to surgery (pre-operative, n=12), and compared to the expression levels of these markers in the same patient's plasma collected three months after curative surgery (post-operative; n=10). It was again reassuring to witness that the expression of each individual miRNA marker exhibited a statistically significantly decrease in expression in the post-operative blood specimens (P<0.05; FIGS. 4A-4D). Finally, in comparison with the risk probability in this subset of patients, these findings were again validated as a significantly reduced risk probability in post-operative vs pre-operative blood specimens was observed (P<0.05; FIG. 4E). These findings indicate that once the EOCRC was surgically resected, the levels of these biomarkers significantly decreased in the systemic circulation in the postoperative blood specimens. Taken together, these findings indicate that the four-miRNA panel is specific for EOCRC and will also have clinical utility potential as a disease-monitoring assay.

The Circulating miRNA Panel has the Potential to Identify Patients with LOCRC

Considering that the age is the key discriminator for patients with EOCRC and LOCRC, the performance of miRNA panel was also evaluated among patients with LOCRC and compared with the age-matched non-disease subjects (>50 years old). Interestingly, it was observed that the miRNA panel can also discriminate patients with LOCRC from age-matched non-disease controls with an AUC of 0.84 (95% CI 0.67-0.89, sensitivity 0.82, specificity 0.88; FIG. 5). Collectively, these findings indicated that this circulating biomarker panel also discriminates individuals with LOCRC from those with no disease.

### DISCUSSION

In contrast to the decreasing trend in CRC-related mortality in adults 50 years or older, the overall incidence and mortality of EOCRC have been steadily rising. To add to this clinical challenge, EOCRC cases are often detected at a more advanced stage, which highlights the need for their early detection in order to improve the overall survival in patients with this disease. Although colonoscopy remains the gold standard for the screening and diagnosis of CRC, it has major disadvantages including its invasive nature, potential risk of complications, and high costs. Furthermore, colonoscopy screening in the United States is often recommended at age 45, which will likely miss diagnosis of a large majority of patients with EOCRC. In view of these limitations, diagnostic approaches that are geared towards the early detection of patients with EOCRC remain an important unmet clinical need. In this effort to implement early detection strategies for EOCRC, the diagnostic modality should preferably be acceptable to healthy individuals, inexpensive, rapid and preferably non-invasive. The present disclosure is a significant step forward in this direction, where the systematic and comprehensive biomarker discovery effort identified and established a circulating miRNA signature that is highly robust in the identification of patients with EOCRC. Furthermore, the evidence that the expression of the discovered biomarkers was significantly reduced in plasma samples from patients in post-surgical settings was provided, thereby indicating that the tumor was indeed the source of these circulating markers in patients with EOCRC.

Currently, several types of non-invasive tests for CRC detection are available for clinical practice. Stool tests, including fecal occult blood tests (FOBT), are commonly used for CRC screening. However, due to the poor sensitivity of older-generation guaiac-based fecal tests for advanced adenomas and cancer, the efficacy of these tests is limited, and increasingly those assays are being replaced by fecal immunochemical tests (FITs) for CRC screening in average risk populations. While the sensitivity of FIT for CRC varies widely (depending upon the cut-off value for the quantitative result), some studies have demonstrated a sensitivity for CRC detection to range between 74-79%. Although FITs is an attractive strategy for current noninvasive CRC screening, in order to overcome the limitations and improve its overall diagnostic accuracy, there is a need to develop additional diagnostic options that support current screening strategies. This study is a significant step forward in this direction, where a four-miRNA panel in blood is reported, which is quite robust for the identification of patients with Stage I/II EOCRC. These findings highlight that the miRNA panel will improve the diagnostic performance of CRC screening that can be complemented with current clinical practice including FITs and will serve as an important non-invasive assay for the identification of patients with EOCRC.

Several previous studies have suggested that EOCRC is a biologically and clinically distinct disease compared to LOCRC. (Refs 6, 40). Current CRC screening strategies mainly focus on LOCRC screening. Interestingly, the miRNA panel described herein was also able to discriminate patients with LOCRC from age-matched non-disease controls with robust AUC values. A possible reason for this is that the older age cohort (≥50 years old) can include relatively younger individuals (early 50s) with CRC who might have developed their disease in their 40s that were potentially EOCRC and would have to be defined as EOCRC patients. Accordingly, the four-miRNA panel described herein can discriminate not only EOCRC patients (<50 years old) in the younger age cohort, but also EOCRC patients (≥50 years old, defined as LOCRC, but developed in their 40s) in the older age cohort. These findings indicate that the miRNA panel described herein will serve as an important biomarker for CRC screening that may be complemented with current LOCRC-focused strategy.

In conclusion, using a systemic discovery approach, the inventors identified and developed a novel miRNA signature for the detection of EOCRC patients. The miRNA panel was successfully validated in two independent patient cohorts and robustly distinguished EOCRC patients from non-disease control samples. This four-miRNA biomarker panel will transform the screening of EOCRC patients and subsequently reduce the mortality rates.

### REFERENCES

1. Siegel R L, Miller K D, Jemal A. Cancer statistics, 2018. C A Cancer J Clin 2018; 68:7-30.
2. Malvezzi M, Carioli G, Bertuccio P, et al. European cancer mortality predictions for the year 2018 with focus on colorectal cancer. Ann Oncol 2018; 29:1016-1022.
3. Holme Ø, Bretthauer M, Fretheim A, et al. Flexible sigmoidoscopy versus faecal occult blood testing for colorectal cancer screening in asymptomatic individuals. Cochrane Database Syst Rev 2013: Cd009259.
4. Siegel R L, Fedewa S A, Anderson W F, et al. Colorectal Cancer Incidence Patterns in the United States, 1974-2013. J Natl Cancer Inst 2017; 109.
5 Welch H G, Robertson D J. Colorectal Cancer on the Decline—Why Screening Can't Explain It All. N Engl J Med 2016; 374:1605-7.
6. Stoffel E M, Murphy C C. Epidemiology and Mechanisms of the Increasing Incidence of Colon and Rectal Cancers in Young Adults. Gastroenterology 2020; 158:341-353.
7 Bailey C E, Hu C Y, You Y N, et al. Increasing disparities in the age-related incidences of colon and rectal cancers in the United States, 1975-2010. JAMA Surg 2015; 150:17-22.
8. Liu P H, Wu K, Ng K, et al. Association of Obesity With Risk of Early-Onset Colorectal Cancer Among Women. JAMA Oncol 2019; 5:37-44.
9. Burnett-Hartman A N, Lee J K, Demb J, et al. An Update on the Epidemiology, Molecular Characterization, Diagnosis, and Screening Strategies for Early-Onset Colorectal Cancer. Gastroenterology 2021; 160:1041-1049.
10. Siegel R L, Miller K D, Goding Sauer A, et al. Colorectal cancer statistics, 2020. C A Cancer J Clin 2020; 70:145-164.
11. Kothari N, Teer J K, Abbott A M, et al. Increased incidence of FBXW7 and POLE proofreading domain mutations in young adult colorectal cancers. Cancer 2016; 122:2828-35.
12. Pearlman R, Frankel W L, Swanson B, et al. Prevalence and Spectrum of Germline Cancer Susceptibility Gene Mutations Among Patients With Early-Onset Colorectal Cancer. JAMA Oncol 2017; 3:464-471.
13. Kirzin S, Marisa L, Guimbaud R, et al. Sporadic early-onset colorectal cancer is a specific sub-type of cancer: a morphological, molecular and genetics study. PLOS One 2014; 9: e103159.
14. Cavestro G M, Mannucci A, Zuppardo R A, et al. Early onset sporadic colorectal cancer: Worrisome trends and oncogenic features. Dig Liver Dis 2018; 50:521-532.
15. Connell L C, Mota J M, Braghiroli M I, et al. The Rising Incidence of Younger Patients With Colorectal Cancer: Questions About Screening, Biology, and Treatment. Curr Treat Options Oncol 2017; 18:23.
16. Zaborowski A M, Abdile A, Adamina M, et al. Characteristics of Early-Onset vs Late-Onset Colorectal Cancer: A Review. JAMA Surg 2021; 156:865-874.
17. Aherne S T, Madden S F, Hughes D J, et al. Circulating miRNAs miR-34a and miR-150 associated with colorectal cancer progression. BMC Cancer 2015; 15:329.
18. Yantiss R K, Goodarzi M, Zhou X K, et al. Clinical, pathologic, and molecular features of early-onset colorectal carcinoma. Am J Surg Pathol 2009; 33:572-82.
19. Goel A, Nagasaka T, Spiegel J, et al. Low frequency of Lynch syndrome among young patients with non-familial colorectal cancer. Clin Gastroenterol Hepatol 2010; 8:966-71.
20. Chang D T, Pai R K, Rybicki L A, et al. Clinicopathologic and molecular features of sporadic early-onset colorectal adenocarcinoma: an adenocarcinoma with frequent signet ring cell differentiation, rectal and sigmoid involvement, and adverse morphologic features. Mod Pathol 2012; 25:1128-39.
21. Wolf A M D, Fontham E T H, Church T R, et al. Colorectal cancer screening for average-risk adults: 2018 guideline update from the American Cancer Society. C A Cancer J Clin 2018; 68:250-281.
22. Sørensen C G, Karlsson W K, Pommergaard H C, et al. The diagnostic accuracy of carcinoembryonic antigen to detect colorectal cancer recurrence—A systematic review. Int J Surg 2016; 25:134-44.
23. Su B B, Shi H, Wan J. Role of serum carcinoembryonic antigen in the detection of colorectal cancer before and after surgical resection. World J Gastroenterol 2012; 18:2121-6.
24. Lee M W, Pourmorady J S, Laine L. Use of Fecal Occult Blood Testing as a Diagnostic Tool for Clinical Indications: A Systematic Review and Meta-Analysis. Am J Gastroenterol 2020; 115:662-670.
25. Lee J K, Liles E G, Bent S, et al. Accuracy of fecal immunochemical tests for colorectal cancer: systematic review and meta-analysis. Ann Intern Med 2014; 160:171.
26. Heitzer E, Haque I S, Roberts C E S, et al. Current and future perspectives of liquid biopsies in genomics-driven oncology. Nat Rev Genet 2019; 20:71-88.
27. Siravegna G, Mussolin B, Venesio T, et al. How liquid biopsies can change clinical practice in oncology. Ann Oncol 2019; 30:1580-1590.
28. Shigeyasu K, Toden S, Zumwalt T J, et al. Emerging Role of MicroRNAs as Liquid Biopsy Biomarkers in Gastrointestinal Cancers. Clin Cancer Res 2017; 23:2391-2399.
29. Hur K, Toiyama Y, Okugawa Y, et al. Circulating microRNA-203 predicts prognosis and metastasis in human colorectal cancer. Gut 2017; 66:654-665.
30. Ozawa T, Kandimalla R, Gao F, et al. A MicroRNA Signature Associated With Metastasis of T1 Colorectal Cancers to Lymph Nodes. Gastroenterology 2018; 154:844-848.e7.
31. Wada Y, Shimada M, Murano T, et al. A Liquid Biopsy Assay for Noninvasive Identification of Lymph Node Metastases in T1 Colorectal Cancer. Gastroenterology 2021; 161:151-162.e1.
32. Bartel D P. MicroRNAs: genomics, biogenesis, mechanism, and function. Cell 2004; 116:281-97.
33. Ng E K, Chong W W, Jin H, et al. Differential expression of microRNAs in plasma of patients with colorectal cancer: a potential marker for colorectal cancer screening. Gut 2009; 58:1375-81.
34. Huang Z, Huang D, Ni S, et al. Plasma microRNAs are promising novel biomarkers for early detection of colorectal cancer. Int J Cancer 2010; 127:118-26.
35. Toiyama Y, Takahashi M, Hur K, et al. Serum miR-21 as a diagnostic and prognostic biomarker in colorectal cancer. J Natl Cancer Inst 2013; 105:849-59.
36. Zanutto S, Ciniselli C M, Belfiore A, et al. Plasma miRNA-based signatures in CRC screening programs. Int J Cancer 2020; 146:1164-1173.
37. Peltier H J, Latham G J. Normalization of microRNA expression levels in quantitative R T-PCR assays: identification of suitable reference RNA targets in normal and cancerous human solid tissues. Rna 2008; 14:844-52.
38. Kandimalla R, Gao F, Matsuyama T, et al. Genome-wide Discovery and Identification of a Novel miRNA Signature for Recurrence Prediction in Stage II and III Colorectal Cancer. Clin Cancer Res 2018; 24:3867-3877.
39. Low E E, Demb J, Liu L, et al. Risk Factors for Early-Onset Colorectal Cancer. Gastroenterology 2020; 159:492-501.e7.
40. Akimoto N, Ugai T, Zhong R, et al. Rising incidence of early-onset colorectal cancer—a call to action. Nat Rev Clin Oncol 2021; 18:230-243.
41. Cross A J, Wooldrage K, Robbins E C, et al. Faecal immunochemical tests (FIT) versus colonoscopy for surveillance after screening and polypectomy: a diagnostic accuracy and cost-effectiveness study. Gut 2019; 68:1642-1652.
42. Lin J S, Piper M A, Perdue L A, et al. Screening for Colorectal Cancer: Updated Evidence Report and Systematic Review for the U S Preventive Services Task Force. Jama 2016; 315:2576-94.
43. Chiu H M, Lee Y C, Tu C H, et al. Association between early stage colon neoplasms and false-negative results from the fecal immunochemical test. Clin Gastroenterol Hepatol 2013; 11:832-8 el-2.
44. Imperiale T F, Ransohoff D F, Itzkowitz S H, et al. Multitarget stool DNA testing for colorectal-cancer screening. N Engl J Med 2014; 370:1287-97.
45. Li M, Wu P, Yang Z, et al. miR-193a-5p promotes pancreatic cancer cell metastasis through SRSF6-mediated alternative splicing of OGDHL and ECM1. Am J Cancer Res 2020; 10:38-59.
46. Zheng G, Zhang G, Zhao Y, et al. Screening of miRNAs as Prognostic Biomarkers for Colon Adenocarcinoma and Biological Function Analysis of Their Target Genes. Front Oncol 2021; 11:560136.
47. Camps C, Buffa F M, Colella S, et al. hsa-miR-210 Is induced by hypoxia and is an independent prognostic factor in breast cancer. Clin Cancer Res 2008; 14:1340-8.
48. Gee H E, Camps C, Buffa F M, et al. hsa-mir-210 is a marker of tumor hypoxia and a prognostic factor in head and neck cancer. Cancer 2010; 116:2148-58.
49. Ho A S, Huang X, Cao H, et al. Circulating miR-210 as a Novel Hypoxia Marker in Pancreatic Cancer. Transl Oncol 2010; 3:109-13.
50. Hong L, Yang J, Han Y, et al. High expression of miR-210 predicts poor survival in patients with breast cancer: a meta-analysis. Gene 2012; 507:135-8.
51. Zhao A, Li G, Péoc'h M, et al. Serum miR-210 as a novel biomarker for molecular diagnosis of clear cell renal cell carcinoma. Exp Mol Pathol 2013; 94:115-20.
52. Wang W, Qu A, Liu W, et al. Circulating miR-210 as a diagnostic and prognostic biomarker for colorectal cancer. Eur J Cancer Care (Engl) 2017; 26.
53. Świtlik W, Karbownik M S, Suwalski M, et al. miR-30a-5p together with miR-210-3p as a promising biomarker for non-small cell lung cancer: A preliminary study. Cancer Biomark 2018; 21:479-488.
54. Pasculli B, Barbano R, Rendina M, et al. Hsa-miR-210-3p expression in breast cancer and its putative association with worse outcome in patients treated with Docetaxel. Sci Rep 2019; 9:14913.
55. Mosakhani N, Sarhadi V K, Borze I, et al. MicroRNA profiling differentiates colorectal cancer according to KRAS status. Genes Chromosomes Cancer 2012; 51:1-9.
56. Mosakhani N, Pazzaglia L, Benassi M S, et al. MicroRNA expression profiles in metastatic and non-metastatic giant cell tumor of bone. Histol Histopathol 2013; 28:671-8.
57. Li W, Chang J, Tong D, et al. Differential microRNA expression profiling in primary tumors and matched liver metastasis of patients with colorectal cancer. Oncotarget 2017; 8:35783-35791.
58. Muti P, Donzelli S, Sacconi A, et al. MiRNA-513a-5p inhibits progesterone receptor expression and constitutes a risk factor for breast cancer: the hOrmone and Diet in the ETiology of breast cancer prospective study. Carcinogenesis 2018; 39:98-108.
59. Li A, Yu J, Kim H, et al. MicroRNA array analysis finds elevated serum miR-1290 accurately distinguishes patients with low-stage pancreatic cancer from healthy and disease controls. Clin Cancer Res 2013; 19:3600-10.
60. Wang Y, Zhao H, Gao X, et al. Identification of a three-miRNA signature as a blood-borne diagnostic marker for early diagnosis of lung adenocarcinoma. Oncotarget 2016; 7:26070-86.

1-21. (canceled)

22. A method of detecting RNA in a patient with colorectal cancer, the method comprising detecting an elevated expression level, relative to a control, of RNA in a biological sample obtained from the patient; wherein the RNA comprises miR-513a-5p, miR-628-3p, miR-193a-5p, miR-210, miR-4304, miR-194-3p, miR-4453, or a combination of two or more thereof.

23. The method of claim 22, wherein the RNA comprises miR-513a-5p, miR-628-3p, miR-193a-5p, miR-210, or a combination of two or more thereof.

24. The method of claim 22, wherein the RNA comprises miR-513a-5p, miR-628-3p, miR-193a-5p, and miR-210.

25. The method of claim 22, wherein the RNA consists of miR-513a-5p, miR-628-3p, miR-193a-5p, and miR-210.

26. The method of claim 22, wherein the RNA comprises miR-513a-5p, miR-628-3p, miR-193a-5p, miR-210, miR-4304, miR-194-3p, and miR-4453.

27. The method of claim 22, wherein the RNA consists of miR-513a-5p, miR-628-3p, miR-193a-5p, miR-210, miR-4304, miR-194-3p, and miR-4453.

28. The method of claim 22, wherein the biological sample is a blood sample.

29. The method of claim 22, wherein the biological sample is a tissue sample.

30. The method of claim 22, further comprising administering to the patient an effective amount of a chemotherapeutic agent.

31. The method of claim 30, wherein the chemotherapeutic agent is an alkylating agent, an antimetabolite compound, an anthracycline compound, an antitumor antibiotic, a platinum compound, a topoisomerase inhibitor, a vinca alkaloid, a taxane compound, an epothilone compound, or a combination of two or more thereof.

32. The method of claim 30, wherein the chemotherapeutic agent is carboplatin, chlorambucil, cyclophosphamide, melphalan, mechlorethamine, procarbazine, thiotepa, azacitidine, capecitabine, cytarabine, gemcitabine, doxifluridine, hydroxyurea, methotrexate, pemetrexed, 6-thioguanine, 5-fluorouracil, 6-mercaptopurine, daunorubicin, doxorubicin, idarubicin, epirubicin, mitoxantrone, actinomycin, bleomycin, mitomycin, valrubicin cisplatin, oxaliplatin, irinotecan, topotecan, amsacrine, etoposide, teniposide, eribulin, vincristine, vinblastine, vinorelbine, vindesine, paclitaxel, docetaxel, epothilone, ixabepilone, patupilone, or sagopilone.

33. A method of treating colorectal cancer in a patient in need thereof, the method comprising:
(i) detecting an elevated expression level, relative to a control, of RNA in a biological sample obtained from the patient; wherein the RNA comprises miR-513a-5p, miR-628-3p, miR-193a-5p, miR-210, miR-4304, miR-194-3p, miR-4453, or a combination of two or more thereof; and
(ii) administering to the patient an effective amount of an anti-cancer agent, administering to the patient an effective amount of radiation therapy, administering to the patient image-based screening, surgically removing all or a portion of the colon of the patient, or a combination of two or more thereof.

34. The method of claim 33, wherein the RNA comprises miR-513a-5p, miR-628-3p, miR-193a-5p, and miR-210.

35. The method of claim 33, wherein the RNA consists of miR-513a-5p, miR-628-3p, miR-193a-5p, and miR-210.

36. The method of claim 33, wherein the RNA comprises miR-513a-5p, miR-628-3p, miR-193a-5p, miR-210, miR-4304, miR-194-3p, and miR-4453.

37. The method of claim 33, wherein the RNA consists of miR-513a-5p, miR-628-3p, miR-193a-5p, miR-210, miR-4304, miR-194-3p, and miR-4453.

38. The method of claim 33, wherein the biological sample is a blood sample or a tissue sample.

39. The method of claim 33, wherein step (ii) comprises administering to the patient an effective amount of a chemotherapeutic agent; wherein the chemotherapeutic agent is an alkylating agent, an antimetabolite compound, an anthracycline compound, an antitumor antibiotic, a platinum compound, a topoisomerase inhibitor, a vinca alkaloid, a taxane compound, an epothilone compound, or a combination of two or more thereof.

40. The method of claim 33, wherein step (ii) comprises administering to the patient an effective amount of a chemotherapeutic agent; wherein the chemotherapeutic agent is carboplatin, chlorambucil, cyclophosphamide, melphalan, mechlorethamine, procarbazine, thiotepa, azacitidine, capecitabine, cytarabine, gemcitabine, doxifluridine, hydroxyurea, methotrexate, pemetrexed, 6-thioguanine, 5-fluorouracil, 6-mercaptopurine, daunorubicin, doxorubicin, idarubicin, epirubicin, mitoxantrone, actinomycin, bleomycin, mitomycin, valrubicin cisplatin, oxaliplatin, irinotecan, topotecan, amsacrine, etoposide, teniposide, eribulin, vincristine, vinblastine, vinorelbine, vindesine, paclitaxel, docetaxel, epothilone, ixabepilone, patupilone, or sagopilone.

41. A kit comprising reagents capable of detecting an expression level of RNA from a biological sample; wherein the RNA comprises miR-513a-5p, miR-628-3p, miR-193a-5p, miR-210, miR-4304, miR-194-3p, miR-4453, or a combination of two or more thereof.
